(19) United States
(12) Patent Application Publication
(10) Pub. No.: US 2021/0071250 A1
(43) Pub. Date: Mar. 11, 2021

(54) DIAGNOSTIC AND PROGNOSTIC LIQUID BIOPSY BIOMARKERS FOR ASTHMA

(71) Applicant: University of Sharjah, Sharjah (AE)

(72) Inventors: Rifat Hamoudi, Sharjah (AE); Qutayba Hamid, Sharjah (AE); Mahmood Hachim, Sharjah (AE); Bassam Mahboub, Sharjah (AE)

(21) Appl. No.: 16/562,861

(22) Filed: Sep. 6, 2019

(51) Int. Cl. C12Q 1/6883; G16H 10/40; G16H 50/30; G16H 50/20

(52) U.S. Cl. CPC C12Q 1/6883; G16H 10/40; C12Q 2600/112; G16H 50/20; C12Q 2600/118; G16H 50/30

(57) ABSTRACT

The invention provides an identification system for ascertaining who is at risk of haying or already has asthma or severe asthma. The system is based upon transcriptomic expression data from RNA extracted from saliva, blood, sputum, bronchial brush, and biopsies samples as well as optionally demographic, risk factor, and symptom data. Also provided are related materials and methods, including such as primers and kits.

### REFERENCE TO AN ELECTRONIC SEQUENCE LISTING

The contents of the electronic sequence listing (Tamimi_19.31_SequenceListing.txt; Size: 1,564 bytes; and Date of Creation: May 13, 2020) is herein incorporated by reference in its entirety.

### TECHNICAL FIELD

The present invention provides an identification system for ascertaining, who is at risk of having asthma or currently has asthma or severe asthma. The system is based on transcriptomic expression data from messenger RNA (mRNA) extracted from saliva, blood, sputum, bronchial brush, and biopsies samples as well patients demographic data like age, sex, ethnicity, asthma risk factors, and clinical and laboratory tests and exam routinely done for asthma diagnosis. Also provided related materials and methods, including primers and kits used.

### BACKGROUND ART

Asthma is one of the most common chronic disease, usually characterized by being a lifelong condition and carrying a high disease burden. Bronchial hyperresponsiveness, inflammation, and airway obstruction episodes are the main characteristic features of this disease. The prevalence of asthma was shown to have increased in the last years, reaching alarming levels. Many theories about the factors that render people at risk of developing asthma were proposed without conclusive results. It is not yet understood what converts fully controlled cases into severe or fatal cases. Asthma is unpredictable, and severity can fluctuate into asthma attack leading to sudden death. Hospital admissions and mortality in individuals with asthma has been rising in the past ten years; nevertheless, new drug discovery has progressed slower than in other specialties. Also, a significant issue that is emerging in asthma is its heterogeneity rather than being a single disease. Such heterogeneity can be attributed to the fact that the airways constrict differently in response to the same provoking stimuli, leading to a heterogeneous clinical presentation that indicates the heterogeneity of the underlying pathogenesis.

Omics technologies such as genomics, transcriptomics, proteomics, and metabolomics, provide extremely detailed molecular-level information and enriched our understanding about the molecular basis of asthma, but fails to elucidate the big picture. Transcriptomic analysis of the airways has the potential to discover gene expression profiles that are characteristic of asthma and has shown promising power to identify different molecular mechanisms that separate different asthmatic phenotypes. Despite all efforts and costs spent on these studies, no conclusive results have been obtained, or results were at sometimes contradictory with each other. The biological processes that underlie complex diseases like asthma do not operate in isolation but manifest collectively as woven intercalated molecular cascades and interactions. The biological complexity of asthma can not be captured using an isolated output from each of those omics technologies.

Biomarkers that aid in asthma phenotyping allows physicians to “personalize” treatment with targeted biological agents. Unfortunately, testing for these biomarkers is not routine in patients with refractory asthma to standard therapy. Scientific advances in recognition of sensitive and specific biomarkers are steadily outpacing the clinical availability of reliable and non-invasive assessments methods designed for the prompt and specific diagnosis, classification, treatment, and monitoring of severe asthma patients. The current diagnosis of asthma through a combination of clinical history with pulmonary function testing and methacholine or exercise challenge test does not explicitly characterize or quantify airway inflammation. Currently, there is a limited set of relevant biomarkers, like eosinophil counts, a fraction of exhaled nitric oxide [Feno] values, and periostin and IgE levels. However, these markers have limitations because they define patients with a Th2 high pattern while a significant proportion of asthmatic patients do not exhibit a Th2 pattern, with the result that this testing does not convey the full story. Therefore, it can be seen that a novel integrated approach that can unravel the complexity of the molecular basis of asthma would contribute the art.

### SUMMARY OF THE EMBODIMENTS

In accordance with the present disclosure, there is provided a technique for assessing salivary and blood biomarkers. Thus, in one aspect there is provided a method for assessing the asthma status of a subject, the method comprising: (a) providing a saliva and blood sample from the subject; (b) assessing the mRNA in the sample to determine the level of transcription of at least one gene selected from Table 1 or Table 2 across different asthma endotypes; (c) utilizing the results of (b) and to characterize the subject according to their probable asthma status. In an embodiment, (d) utilizing the characterization of the subject according to their probable asthma status to establish a diagnostic and prognostic intervention plan and/or treatment for the subject.

As well as providing diagnostic information about the subject per se, in another embodiment, the method may be used to stratify a population according to their relative risk. This allows health providers to prioritize further actions. It should be understood that “risk” in this context is used in a relative context with respect to a population of which the subject is a member. Thus, in an additional embodiment, the method may be used for assessing the asthma risk status for a subject.

In accordance with a second aspect of the present disclosure, there is provided a method that may be used within a population to establish asthma development risk in the members of the population and/or determine who is at higher risk of developing asthma or severe asthma.

In accordance with a third aspect of the present disclosure, there is provided a method that may be used to classify healthy subjects into category 1, characterized as low risk, category 2 as medium risk, and category 3 as high risk of developing asthma. In a preferred embodiment the method may be used to diagnostically characterize the subject according to the following asthma severity: (1) healthy; (2) mild asthma; (3) moderate asthma, and (4) severe asthma.

In some embodiments, the method is used for assessing the risk of developing severe asthma in an asthmatic subject.

Thus, in accordance with a fourth aspect of the present disclosure and without limitation, the method may be used for one or more of
diagnosing or aiding in the diagnosis of asthma and asthma severity in a subject
assessing the risk of developing asthma in the subject
prognosis or aiding in the prognosis of asthma in the subject
assessing the efficacy of a treatment for asthma in the subject
monitoring the progression or regression of asthma in the subject
developing novel risk prediction model based on the RNA salivary markers described herein.

In preferred embodiments, the gene expression of Table 1 has been utilized by the inventors as significant Differently Expressed Genes (DEG) in healthy and asthmatic patients useful to determine the asthma status of a subject.

Transcriptome Biomarkers

As used herein, “transcriptomic based biomarker” refers to one of the genes listed in Table 1 and Table 2 and Appendix—Table 6.

### BRIEF DESCRIPTION OF THE FIGURES

FIG. 1 is a flowchart illustrating a method for raw CEL files processing, normalization, and filtration.

FIG. 2A, FIG. 2B, FIG. 2C, and FIG. 2D illustrate the results of a Gene Set Enrichment to identify genes differentially expressed between healthy vs. mild bronchial epithelium (FIG. 2A), healthy vs. moderate bronchial epithelium (FIG. 2B), mild vs. moderate bronchial epithelium (FIG. 2C), and moderate vs. severe bronchial epithelium (FIG. 2D).

FIG. 3 is a Venn diagram illustrating the relative sizes and extent of overlap of gene subsets identified in the Gene Set Enrichment of FIG. 7.

FIG. 4 illustrates differential mRNA expression level using RT qPCR for genes related to cell cycle, proliferation, cell division and DNA synthesis in primary cells from healthy versus asthmatic cells from lung epithelial, fibroblasts, and PBMC.

FIG. 5 is a chart showing differential mRNA expression level using RT qPCR in different diseases stages in healthy (n=3), mild asthmatic (n=3), and severe asthmatic and upregulation of CCND2 compared to other groups in larger sets of bronchial fibroblasts for genes related to cell cycle, proliferation, cell division and DNA synthesis in primary cells from healthy versus asthmatic cells.

FIG. 6 includes graphs showing mRNA expression level using RT qPCR in PBMC from healthy (n=10) versus mild to moderate asthmatic (n=10), and severely asthmatic patients (n=9).

FIG. 7 is an ELISA showing AREG protein level from plasma and saliva samples from healthy, mild to moderate, and severe asthma. AREG protein level can discriminate healthy from mild to moderate and Severe Asthma in plasma and saliva using ELISA.

FIG. 8 includes charts illustrating AREG, FOSL1, SFN mRNA expression in PBMC cutoff values from healthy and asthmatic samples. AREG, FOSL1, SFN mRNA expression in PBMC can differentiate asthmatic from healthy controls. Their expression can predict the severity of asthma.

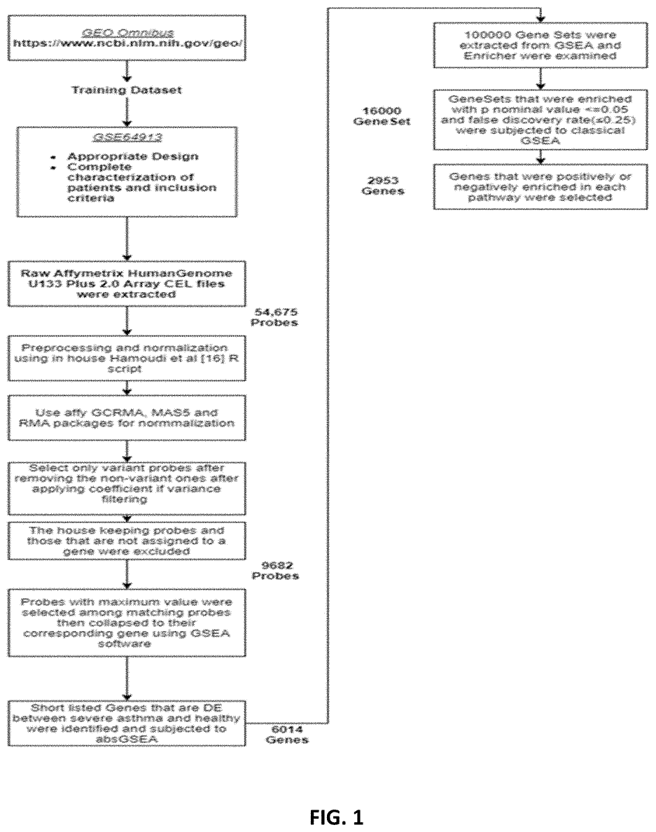

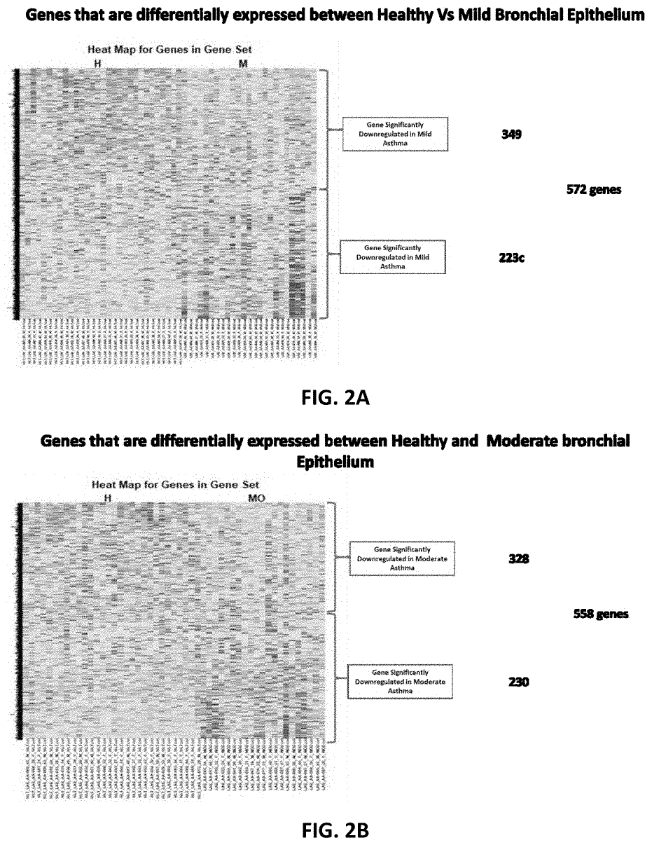

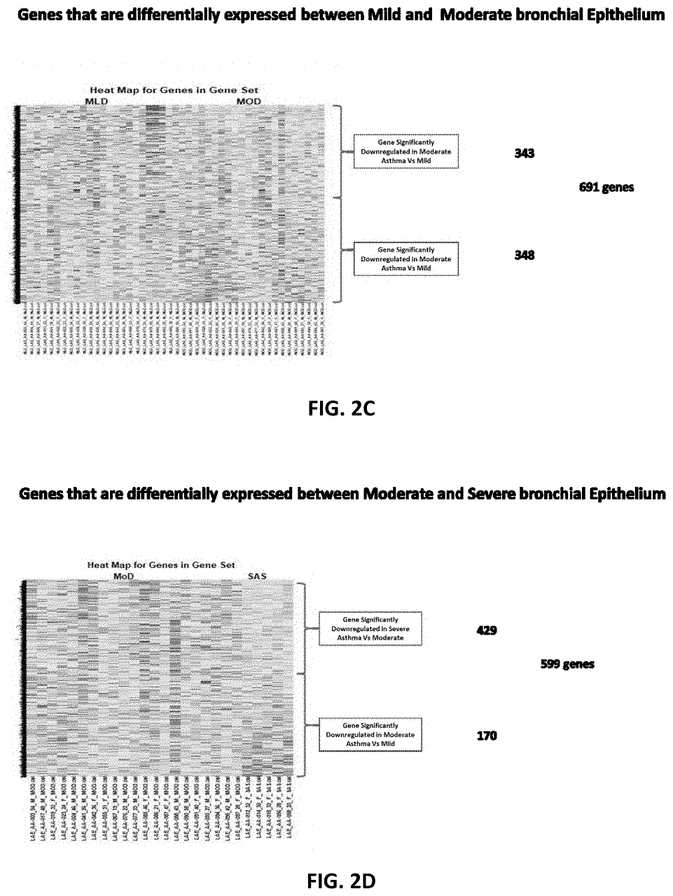

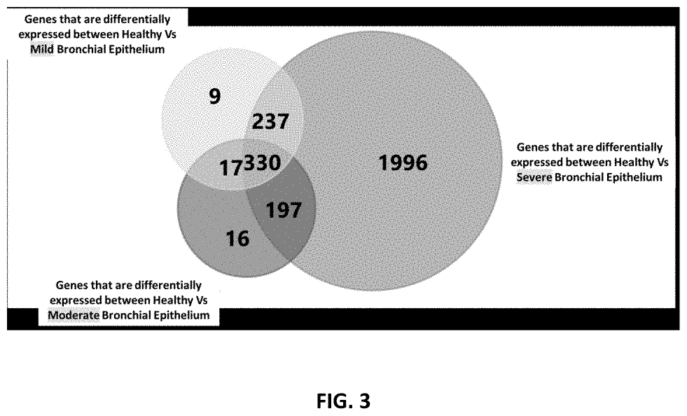

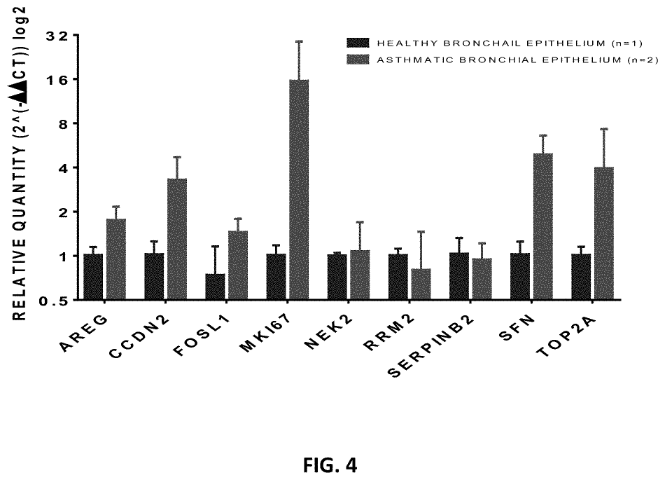

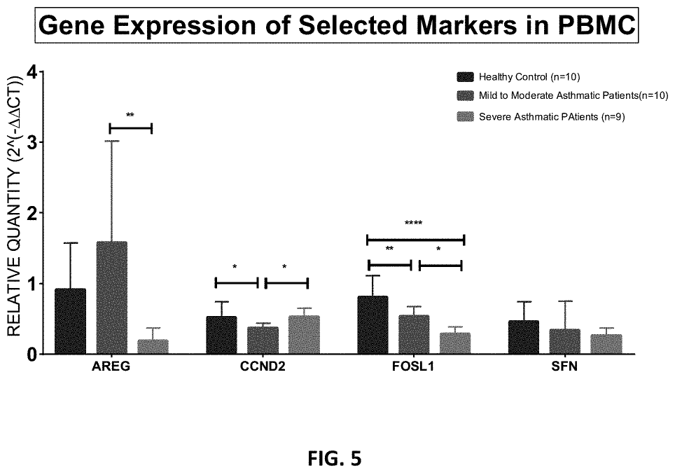

### DETAILED DESCRIPTION OF SPECIFIC EMBODIMENTS

The present invention provides an identification system for ascertaining, who is at risk of having asthma or currently has asthma or severe asthma. The system is based on transcriptomic expression data from mRNA extracted from bronchial biopsies, brush and liquid biopsies such as saliva and blood samples. Optionally, this can be combined together with patients' demographic data such as age, sex, ethnicity, asthma risk factors, which can be collected using a simple questionnaire, in addition to clinical exam and laboratory tests routinely carried out for asthmatic patients. The biomarkers identified can differentiate mild, moderate, and severe asthma, which is not yet available in clinical practice. Since the inventors utilize publicly available data, they were able to determine the validity of genes identified in more than 7,000 samples available, providing valid and robust support to the results obtained using this approach.

Briefly, the inventors utilized publicly available transcriptomics of Bronchial Epithelium, demographic, symptom, and risk factor data from 1,000 patients. These data have been analyzed using a novel artificial intelligence technique. From this, they identified a list of 76 genes differentially expressed between asthmatic (mild, moderate, and severe) compared to healthy control. Nine genes were specific to middle asthma compared to healthy, and 16 genes were specific to moderate asthma compared to healthy. Another list of 225 gene set of RNA markers that differentiate severe asthmatic from healthy and another asthmatic (mild and moderate).

The inventors further used the gene signature identified in gene expression analysis system to profile 60 distinct human RNA targets using a highly multiplexed amplification method in a further analysis data of RNA extracted from saliva, sputum, blood bronchial brush, and biopsies samples as well as optionally demographic, risk factor, and symptom data which can be collected using a sample questionnaire. The cohort used for validation was locally recruited, 60 patients, 20 normal, 20 nonsevere mild to moderate asthma, and 20 with severe asthma. The analysis has shown that combining just a small selection of RNA amplicons with simple demographic data and routine lab test like Peak flow, a blood differential count can accurately characterize (or classify, the terms are used interchangeably) the asthmatic status of a subject and identify those with or at risk of progressing to severe asthma and those that will not respond to asthma-specific treatments.

Saliva collection is easy, innocuous, acceptable by patients, and thus it represents a potential tool for measuring disease biomarkers with the potential to reduce the requirement for costly and invasive bronchoscopic investigations in subjects who are at risk of developing asthma, specifically severe asthma. Circulating genes expression level can be a reliable, non-invasive, and cost-effective biomarker that can provide additional discriminating power to the available clinical and laboratory tests of severe asthma.

Datasets Selection.

The methods described can provide information regarding healthy versus asthmatic sample. In one embodiment the method may provide information to identify DEG in acute asthmatic attack subjects versus convalescent phase, whether the sample taken is from bronchial biopsy versus brush or liquid biopsy, and the DEG in PBMC in response to the most common asthma allergen. Thus, the methods of the present invention can provide useful information in identifying DEG in severe and moderate asthmatic bronchial biopsy, brush, PBMC and saliva tissue.

In some embodiments, the subject may be identified as a candidate for being at risk of developing asthma or severe asthma. The methods disclosed herein are useful because they enable health care providers to determine appropriate diagnostic intervention and/or treatment plans. In one embodiment, the characterization of the subject as being at risk of developing (2) mild asthma may be used to decide a non-urgent further diagnostic intervention is required. In another embodiment, the characterization of the subject as being at risk of developing (3) moderate or (4) severe asthma may be used to decide an urgent further diagnostic intervention is required.

As shown in the examples, in preferred embodiments and based on the dataset herein, the system can provide clinically relevant limits of sensitivity and specificity. As described in more detail hereinafter, the choice of markers or other expression limits provide combinations of sensitivity and specificity as desired.

“Diagnostic” in this context means identifying the presence or nature of a pathological condition such as asthma. Diagnostic methods differ in their sensitivity and specificity. The “sensitivity” of a diagnostic test is the percentage of diseased who test positive (percent of “true positives”). Subjects who are not diseased and who test negative in the assay, are termed “true negatives.” Diseased individuals not detected by the assay are “false negatives.” The specificity of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

Choice of the Subject.

The term “subject,” as used herein, generally refers to a mammal. Typically the subject is a human. Where the present invention relates to the analysis of nucleic acid of a subject, such an individual may be entirely symptomless or maybe one who has asthma. A subject under the care of a physician or other health care provider may be referred to as a “patient.”

The method may be used to assess risk within a population by screening individual members of that population.

The Use of Saliva is Preferred in the Present Invention.

Saliva for use in the invention can be simply and acceptable collected and easily stored using conventional techniques. Increasing bodies of work are utilizing saliva and are detecting useful biomarkers in asthma studies.

Methods of the present invention may include obtaining a saliva sample comprising nucleic acid from an individual. Alternatively, the assessment of the biomarkers herein may be performed or based on a historical sample, or information already obtained therefrom.

In some embodiments, a saliva sample was collected from subjects who were asked to fast without eating or drinking for 1 hour before the saliva collection by gargling and rinsing the mouth with water 5 minutes before proceeding with the saliva collection. 1 mL of unstimulated whole saliva via passive drool was collected in pre-prepared 50 mL tube containing 1 mL of RNAlater (Invitrogen).

Specifically, the methods may optionally involve obtaining a saliva sample from a subject. As used herein, the term “obtaining a saliva sample” refers to any process for directly or indirectly acquiring the saliva sample from a subject. For example, a saliva sample may be obtained (e.g., at a laboratory facility) from one or more persons who procured the sample directly from the subject.

In some embodiments, the use of blood or the use of sputum, or the use of bronchial biopsy sample from a subject may be utilized in the present invention.

Datasets Selection.

Datasets to Identify Common DEG in Bronchial Epithelium of Asthmatic Patients.

The methods of the present invention utilized the publicly available transcriptomic dataset extracted from Gene Expression Omnibus (GEO) of asthmatic patients. The inclusion criteria for selecting datasets were as follows: datasets on human samples only, an experiment that included matching healthy control, only datasets with defined clinical classifications of participants, only dataset with bronchial epithelium gene expression using microarray. The inventors used dataset (GSE64913) as training dataset because it is designed with a complete characterization of patients and inclusion criteria. Out of the total 70 samples included in those studies, 33 asthmatic patients were compared to 37 healthy controls, as shown in Table 1.

TABLE 1
Details of Datasets extracted from the Gene Expression
Omnibus (GEO) used as a training set in the study

| | | | Severe | | |
|---|---|---|---|---|---|
| GeoID | Title | Total | Asthma | Moderate | Healthy |
| GSE64913 | Altered epithelial gene expression in | 70 | 33 | — | 37 |
| | peripheral airways of severe asthma | | | | |
| GSE76227 | Expression data of bronchial biopsies and | 190 | 121 | 69 | |
| | epithelial brushing from Unbiased | | | | |
| | Biomarkers in Prediction of REspiratory | | | | |
| | Disease outcomes (U-BIOPRED) Project | | | | |
| GSE27335 | Genomic differences distinguish the | 24 | 16 | | 8 |
| | myofibroblast phenotype of the distal lung | | | | |
| | from airway fibroblasts | | | | |
| GSE31773 | Comparison of mRNA expression in | 40 | 16 | 8 | 16 |
| | circulating T-cells from patients with | | | | |
| | severe asthma | | | | |
| GSE16032 | Gene expression data from severe asthmatic | 25 | | | |
| | children: PBMC profiles during acute | | | | |
| | exacerbation versus convalescence | | | | |
| GSE73482 | Gene expression patterns in allergen-driven | 72 | | | |
| | CD4 T cell responses from human topics | | | | |
| | with or without asthma. | | | | |

Dataset to Identify DEG in Severe and Moderate Asthmatic Bronchial Biopsy Versus Brush.

Another dataset (GSE76227) with 190 samples (121 severe asthmatic and 69 moderate ones) where bronchial epithelial brush transcriptomics was compared to bronchial biopsy was used to find the identified genes are capable of detecting the disease even when the sample contains other than epithelial cells.

Dataset to Identify DEG in Asthmatic Lung Fibroblasts Compared to Healthy Fibroblasts in Different Locations of the Lung.

The GSE27335 dataset was used in the present invention to determine the DEG between bronchial and parenchymal fibroblasts in healthy and asthmatic patients.

Dataset to Identify DEG in Severe Versus Non-Severe Asthmatic PBMC Compared to Healthy.

Three datasets were used in the present invention to identify DEG in PBMC in healthy versus asthmatic samples in each cell type (CD4 vs. CD8 T lymphocytes) (GSE73482), and DEG in atopic asthmatic patients in acute versus convalescent asthma attack (GSE16032). A third dataset (GSE73482) was used to identify the DEG in PBMC in response to the most common asthma allergen (house dust mite).

Validation of Common DEG in Bronchial Epithelium of Asthmatic Patients and Deciphering Their Profiles in Conditions Other Than Asthma.

The methods of the present inventions extracted seven datasets of the same microarray platform, representing different types of samples other bronchial epithelium (nasal scraping, sputum, and blood) as well as different variables such as smoking, steroid inhaler treatment, acute versus convalescent conditions, rhinovirus infection, exercise-induced bronchospasm, to identify the variables that may affect their expression. The inventors compared the different locations of airway epithelium (nasal, central airway and peripheral), gender-specific variations, and looked for other respiratory diseases that share common features with asthma, like COPD and IPF. The total number of explored samples was 615 (263 asthmatic, 36 COPD, 23 IPF, 60 smokers, and 184 healthy controls), as shown in Table 2:

TABLE 2
Details of Datasets extracted from Gene Expression Omnibus (GEO) used as a validation set in the study
Validation sets

| Geo ID | Title | Total | Asthmatic | COPD | IPF | Smoker | Healthy |
|---|---|---|---|---|---|---|---|
| GSE41861 | Upper airway gene expression is an | 138 | 54 | | | | 30 |
| | effective surrogate biomarker for | | | | | | |
| | Th2-driven inflammation in the | | | | | | |
| | lower airway | | | | | | |
| GSE41862 | Nasal scrape gene expression profiling | 116 | 95 | | | | 21 |
| | in asthmatics | | | | | | |
| GSE16032 | Gene expression data from severe | 10 | 25 | | | | |
| | asthmatic children: PBMC profiles | | | | | | |
| | during acute exacerbation versus | | | | | | |
| | convalescence | | | | | | |
| GSE35571 | Gene expression data from 131 human | 131 | 67 | | | | 64 |
| | subjects in Detroit, Michigan | | | | | | |
| GSE13785 | Novel mediators of eicosanoid and | 22 | 22 | | | | |
| | epithelial nitric oxide production | | | | | | |
| | in asthma | | | | | | |
| GSE30063 | Epithelial Expression of Toll-like | 169 | | 36 | | 60 | 63 |
| | Receptor 5 is Modulated in Healthy | | | | | | |
| | Smokers and Smokers with Chronic | | | | | | |
| | Obstructive Lung Disease | | | | | | |
| GSE21369 | Gene expression profiles of | 29 | | | 23 | | 6 |
| | interstitial lung disease | | | | | | |
| | (ILD) patients | | | | | | |
| Total | 7 | 615 | 263 | 36 | 23 | 60 | 184 |

TABLE 2a
List of forward and reverse primers for each of the genes
assessed by qRT-PCR.

| | SEQ | NCBI | Forward Primer | Reverse Primer | Amplicon |
|---|---|---|---|---|---|
| Gene | ID. NO. | Ref. Seq. | Sequence (5′-3′) | Sequence (5′-3′) | Size (bp) |
| ARE G | 1 | NM_001657.4 | GAGCACCTGGAAGCAGTAAC | GGATCAGCAGACATAAAGGC | 151 |
| MKI67 | 2 | NM_002417.5 | GAAGAGCTCCTAGCAGTCG | GGCCACTTCTTCATTCCAG | 161 |
| NEK2 | 3 | NM_002497.4 | TATTGTGAAGGAGGGGATCTG | CGATGCAATACGGTATGACC | 158 |
| | | | | AC | |
| RRM2 | 4 | NM_001034.4 | GCCATTGAAACGATHCCTTG | GCAAAGGCTACAACACGTTC | 101 |
| SFN | 5 | NM_006142.5 | CGACAAGAAGCGCATCAT | GTGGTCTTGGCCAGAGAG | 177 |
| TOP2A | 6 | NM_001067.4 | GCCCCAAAAGGAACTAAAAG | GGATTTCTTGCTTGTGACTGC | 165 |
| | | | G | | |
| SERPINE1 | 7 | NM_000602.4 | CGCCAGAGCAGGACGAA | GGACACATCTGCATCCTGAA | 65 |
| | | | | GT | |
| CTNNB1 | 8 | NM_001904.4 | CTTACACCCACCATCCCACT | CCTCCACAAATTGCTGCTGT | 197 |
| 18SrNA | 9 | NR_145820.1 | TGACTCAACACGGGAAACC | — | 114 |

Raw CEL Files Processing, Normalization, and Filtration.

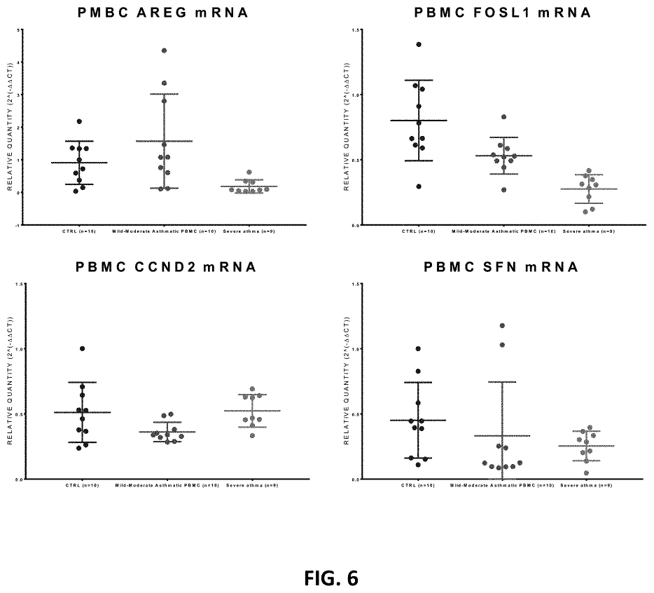

An example method of the present invention extracted files from the Raw Affymetrix Human Genome U133 Plus 2.0 Array CEL. Each dataset underwent preprocessing and normalization separately using in-house R script on R statistical software version 3.02 that uses affy packages GCRMA, MASS, and RMA as shown in the flowchart of FIG. 6. Non-specific filtration approach based on the coefficient of variance was used to exclude non-variant probes. The housekeeping probes and those that were not assigned a gene were excluded, and the resulting filtered probes left were the only variant probes. The datasets with different platforms (GSE27335, Agilent-014850 Whole Human Genome Microarray 4×44K G4112F), and (GSE73482, Affymetrix Human Gene 1,0 ST Array) were processed and normalized using AltAnalyze Software.

Measuring Levels of RNA.

Methods of the invention will generally employ protocols which examine the presence and/or expression of mRNAs, in saliva, or sputum, or blood, or bronchial biopsy sample. Methods for the evaluation of mRNAs in cells are well known.

Probes Collapsing to Their Corresponding Genes.

Probes for the present invention were selected among matching probes, then collapsed to their corresponding gene using GSEA software. The resulting gene list was used for AbsGSEA to identify the significantly enriched pathways. A total of 100,000 gene sets were analyzed, and results were ranked according to the nominal P-value (<0.05) and false discovery rate (≤0.25) as previously described. The gene sets that passed AbsGSEA filtering was explored using classical GSEA to identify genes that positively or negatively enriched in each pathway. Genes that are enriched in more than 100 pathways were selected. For the gene sets differentially regulated between healthy and severe asthma, the leading-edge analysis was performed to identify the biologically important gene subset. A shortlist of 35 genes was identified. The flowchart of the approach is shown in FIG. 6.

Identifying the Confounding Factors.

From healthy group transcriptomic data, the inventors identified genes that are differentially expressed between males and females, large airway and small airway, in order to identify genes that differ due to the disease (severe versus healthy). Another data set was used to differentiate genes that are enriched in the epithelial brush from those that can be also enriched in the bronchial biopsy. The effect of the cell examined whether CD4 or CD8 lymphocytes during an acute attack or in a convalescent-phase were also identified. Individual sample raw mRNA expression samples for the identified genes were extracted from each dataset to confirm the findings and identify the factors that affect its level. For statistical purposes, the inventors used the Student T-test between asthma and control for the gene expression assuming p-value <0.05 as significant.

The methods of the present disclosure may utilize one or more non-genetic factors or markers to assess the status of the individual. “Non-genetic” in this context means not based directly on a nucleic acid-based assessment, but rather relating to other demographic, risk factor or symptom characteristics demonstrated by the subject. These criteria can typically be self-reported using a simple questionnaire.

Patient Selection.

The methods of the present disclosure utilized the collection of blood samples from healthy individuals and from asthmatic patients. Blood from 10 non-severe asthmatic patients (mild to moderate asthma) and from 10 severe asthmatic patients (fulfilling the criteria for asthma as per American Thoracic Society). Those patients were compared to 10 non-asthmatic volunteer subjects who had no recent infection of the respiratory tract and no history of allergy or asthma. Both patients and healthy controls were subjects were recruited in the Asthma Clinic in Rashid Hospital—Pulmonary medicine Department. The study was approved by the Ethics Committee of Dubai Health Authority and the University of Sharjah, and each subject gave written informed consent after a thorough explanation by the treating physicians and the researchers.

Blood samples (12 mL) utilized in the methods of the present invention were collected from each individual in EDTA-containing blood collection tubes (3 mL each) and transferred within two hours to the Sharjah Institute for Medical Research (SIMR) and PBMCs were isolated using techniques well known in the art. Twelve milliliters of Histopaque-1077 (Sigma, #10771, Germany) were added to a 50 mL centrifuge tube and brought to room temperature, then 12 mL of whole blood were carefully layered on top of the Histopaque and centrifuged at 400×g for precisely 30 minutes at room temperature. After centrifugation, the buffy layer interface was carefully collected with a Pasteur pipette and transferred to a clean 15 mL conical centrifuge tube. Subsequently, the cells were washed twice with isotonic phosphate-buffered saline solution and centrifuged at 250×g for minutes, after which the cell pellets were stored at −80+C. until analysis for protein, DNA and RNA extraction.

Any RNA isolation technique that does not select against the isolation of mRNA can be utilized for the purification of RNA from saliva. RNA was extracted using RNAeasy mini kit (Qiagen, #74106, Germany) as per manufacturer instructions. PBMC pellets were lysed first then passed through Qiagen QIAshredder columns (Qiagen, #79656, Germany) for homogenizing the lysates. RNA purity (OD260/280) and quantity were measured using Nanodrop 2000 (Thermo Scientific™, USA). Then, the purified RNA was reverse transcribed into cDNA using High Capacity cDNA Reverse Transcription (Applied Biosystems, #4375222, USA) as per manufacturer instructions. 5× Hot FIREPol EvaGreen qPCR Supermix (Solis BioDyne, Estonia) was used to quantify mRNA of the selected genes using QuantStudio3 (Applied Biosystems, USA).

Primer Design

The target amplicon was chosen based on a unique protein domain to the gene of interest using SMART (Simple Modular Architecture Research Tool). The specific protein domain sequence was back-translated to its corresponding nucleotide sequence using the Consensus CDS (CCDS) database. Then, the identified nucleotide sequence was used for primer design in Primer3 platform. The primers were subjected to in silico QC check for possible dimers. Thermodynamics for the RNA secondary structure of the amplicons were tested using MFOLD tool. The primers were examined for their performance using PCR, agarose gel electrophoresis and melt curves parameters in qRT-PCR.

Transcriptomic Analysis.

The targeted RNA-seq library preparation for the present invention was carried out using AmpliSeq (Thermo Fisher Scientific), which is designed over 21,000 distinct human RNA targets using a highly multiplexed amplification method. Each amplicon represents a unique gene. The average size of each amplicon is ˜15 bp. For library preparation, a barcoded cDNA library was first generated with SuperScript VILO cDNA Synthesis kit from 20 ng of total RNA treated with Turbo DNase (ThermoFisher Scientific). Then cDNA was amplified using Ion AmpliSeq technology to accurately maintain expression levels of all targeted genes. Amplified cDNA libraries were evaluated for quality and quantified using Agilent Bioanalyzer high sensitivity chip. Libraries were then diluted to 100 pM and pooled equally with two individual samples per pool. Pooled libraries were amplified using emulsion PCR on Ion Torrent OneTouch2 instruments (OT2) and enriched following manufacturer's instructions. Templated libraries were then sequenced on an Ion Torrent Proton sequencing system, using Ion PI kit and chip version 2.

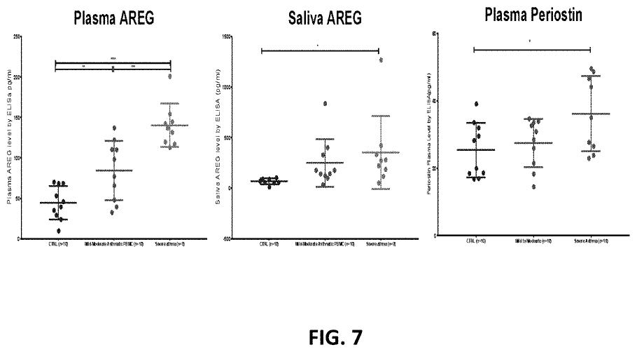

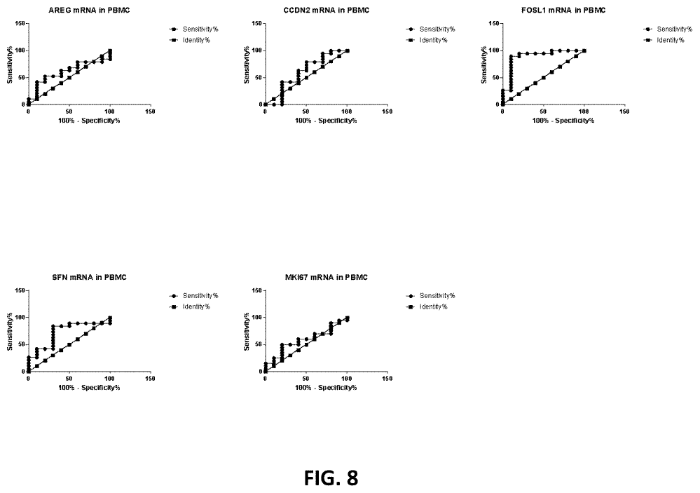

The Resultant 2953 were used as a Gene Set to perform Gene Set Enrichment on a different Dataset to identify genes that are differentially expressed between Mild, Moderate and Severe Asthma as compared to the healthy epithelium (FIG. 7). The Venn diagram of FIG. 8 illustrates the relative sizes and extent of overlap of gene subsets identified in the Gene Set Enrichment. Of the 1996 genes identified as specific to Severe Asthma as compared to the healthy epithelium, 225 showed significant enrichment in more than 50 pathways and were selected as asthma-related genes as compared to healthy control. Of the 330 genes differentiating mild, moderate, and severe from healthy bronchial epithelium, 76 showed significant enrichment in more than 50 pathways and were selected as asthma-related genes compared to healthy control (Table 1).

Targeted RNA-seq using Ion AmpliSeq sequencing analysis of all samples was performed using the Ion Torrent Software Suite version 5.4. The alignment was carried out using the Torrent Mapping Alignment Program (TMAP). TMAP is optimized for Ion Torrent sequencing data for aligning the raw sequencing reads against reference sequence derived from hg19 (GRCh37) assembly. To maintain specificity and sensitivity, TMAP implements a two-stage mapping approach. First, four alignment algorithms, BWA-short, BWA-long, SSHA, and SUper-maximal Exact Matching were employed to identify a list of candidate mapping locations. A further alignment process is performed using the Smith-Waterman algorithm to find the final best mapping. Raw read counts of the targeted genes were performed using samtools (samtools view -c -F 4 -L bed_file bam_file). The quality control, including the number of expressed transcripts, is checked after Fragments Per Kilobase Million (FPKM) normalization. Differentially expressed gene (DEG) analysis was performed using R/Bioconductor package DESeq2 with raw read counts from RNASeq and AmpliSeq. Read count normalization was performed using the regularized logarithm (rlog) method provided in DESeq2. Genes with less than ten normalized read counts were excluded from further analysis. DEG determination was carried out using the LIMMA package.

TABLE 3
76 Genes that Differentiate Asthmatic (mild, moderate
and severe) from Healthy Bronchial Epithelium

| | | | |
|---|---|---|---|
| CD44 | FAM83D | PCDH17 | TMEM45A |
| FOSL1 | FBN2 | PLK4 | UHRF1 |
| KLF4 | FMN2 | PNMA2 | WIF1 |
| RRM2 | FST | POSTN | |
| TOP2A | FXYD6 | PTGFR | |
| PEG10 | GNG2 | RAB6B | |
| AKR1B10 | GSN | RPL41 | |
| ANLN | HOMER2 | RPRM | |
| ARNTL2 | HSPA12A | RTN1 | |
| B4GALT6 | IGFBP5 | SCEL | |
| BCL2L14 | IRS2 | SCG2 | |
| CALD1 | ITPR1 | SERPINB2 | |
| CD109 | JUNB | SERPINB8 | |
| CDH2 | KCNA1 | SGCE | |
| CEL | KIF20A | SHH | |
| CLASP2 | KIT | SLC16A6 | |
| CXCL2 | KRT17 | SLC1A1 | |
| DPYSL2 | LSS | SLC5A1 | |
| DPYSL3 | MAP2 | SLC7A11 | |
| EFHD1 | MATN2 | SOX9 | |
| EFNB2 | NMU | SPP1 | |
| EGR1 | NPAS2 | TACR1 | |
| EPB41L2 | NTRK2 | TBX1 | |
| FAM110C | P4HA2 | TM4SF1 | |

TABLE 4
225 Genes that Differentiate Severe Asthmatic from Healthy and
other asthmatic (mild and moderate) Bronchial Epithelium

| | | | | | | | | |
|---|---|---|---|---|---|---|---|---|
| SERPINB5 | FHL1 | KRT8 | SOX5 | TMEM2 | ADAM19 | RERE | CTNNB1 | LONRF1 |
| IL1RN | PPARGC1A | SPAG5 | TIMP1 | ANXA2 | KCNK5 | GPM6B | BCL11A | TMOD2 |
| GPRC5A | CTNS | CTPS | SLC25A20 | KIAA0101 | ALK | DECR1 | AADAT | IFIT1 |
| SFN | FHL2 | ANKRD28 | CKS2 | DAAM1 | DLX4 | HSPA2 | TCF20 | KLK10 |
| MKI67 | SLC25A25 | ARNTL | F5 | ZWINT | PPARG | EFNA5 | EML4 | FRAS1 |
| AXIN2 | COBL | UST | PALLD | CCNB1 | PDE4D | SLC1A4 | CYLD | CREBBP |
| GSTT1 | GDF15 | PSCA | P2RY1 | ZNF165 | BAG1 | SDK1 | RIF1 | EIF4ENIF1 |
| ZBTB16 | KRT4 | PHLDA1 | SAMHD1 | HSPA5 | KIF4A | DOCK7 | BCL2 | ARNT |
| AREG | EHD2 | BLM | UCK2 | KRT7 | CPE | ACSL4 | ALDH6A1 | RNF152 |
| KRT23 | KLHL18 | TACSTD2 | TNC | MYC | GAPDH | LMO4 | SPON1 | KIF5C |
| TFCP2L1 | HSPA6 | LMNA | TJP1 | KRT6B | DUSP1 | CHL1 | CCND2 | MAOB |
| DLG4 | HPGD | PTRF | CLCA4 | CDC20 | F2RL1 | RBBP4 | ERC1 | CHGB |
| S100A10 | HBEGF | PDK4 | IRF7 | YRDC | RORA | ELL2 | FTO | MTCH2 |
| TRIM7 | HK2 | PLCB1 | GPT2 | CXCL10 | HMGCS1 | ADCY2 | AQP4 | NOTCH2 |
| PTGS2 | MLLT4 | SRD5A1 | BMP2 | CENPF | AFF4 | ZRANB2 | CLK4 | RIMS1 |
| SDPR | ETV1 | CSTA | AURKA | NFKBIA | ISL1 | PTPRZ1 | LSM14A | ITPR2 |
| TUBB6 | FKBP11 | HN1 | ECT2 | RPL28 | CANX | ETNK1 | AKAP11 | P2RY2 |
| S100A8 | ANXA1 | GGH | PPAN | RGS2 | NMNAT2 | PLK2 | AKAP7 | GATM |
| SLC6A8 | SCNN1A | PTP4A1 | TACC1 | KLF6 | BCL9 | AR | NFATC2 | HMGA2 |
| ADAMTS9 | TPX2 | ME1 | CHAF1A | PYGB | CFTR | CAMK2D | BCL11B | GIT2 |
| PKP2 | SLC7A8 | NUFIP2 | GLRX | SYN2 | MYO5A | AASS | STAT1 | WWC2 |
| MTSS1 | BIRC5 | FA2H | H2AFX | IER2 | NCDN | FGFR2 | VANGL2 | WNT5A |
| GAS2L1 | KLF9 | CLTB | TMED9 | RAB40C | SLC6A20 | BRWD1 | FADS1 | RGS17 |
| S100A16 | BCAT1 | MUC6 | PSPH | GRM7 | SATB1 | PKN2 | LYN | PLAG1 |
| SULT2B1 | ITGA6 | RUNX2 | BCR | PERP | ATP1B1 | PARVA | FUBP1 | |

TABLE 5
AREG, FOSL1, SFN mRNA expression in PBMC cutoff values
that can differentiate asthmatic from healthy controls.
Their expression can predict the severity of asthma.

| | Control | Mild Vs Severe | |
|---|---|---|---|
| Cut off Value | vs asthma | Asthma | Control Vs Mils |
| PBMC AREG (RQ) | <0.1324 | <0.09815 | >1.037 |
| PBMC FOSL1 (RQ) | <0.588 | <0.4286 | <0.588 |
| PBMC SFN (RQ) | <0.1455 | >0.2692 | <0.139 |
| PMBC CCND2 (RQ) | <0.3576 | >0.3961 | >73.9 |
| Plasma AREG (μg/ml) | >73.9 | >127.2 | >32.42 |
| Plasma POSTN (μg/ml) | >33.66 | >34.94 | <0.3599 |
| Saliva AREG (μg/ml) | >112.7 | >184.7 | >115 |

APPENDIX
Appendix Table 6: List of the Genes Differentially
expressed in the bronchial epithelium of asthmatic
patients compared to healthy controls

| Control Vs | Control Vs | Control Vs |
|---|---|---|
| Mild Asthma | Moderate Asthma | Severe Asthma |
| VGLL3 | NAPSB | PHACTR3 |
| FHOD3 | NLF2 | CXCL14 |
| WIF1 | SLC1A1 | CD109 |
| PRPF18 | SNX10 | DNAJC12 |
| LOC285419 | SEC14L5 | FKBP5 |
| PEG3 | ZNF663 | MUC12 |
| PNMA2 | ARID5B | TPRXL |
| RUNX1T1 | LOC221442 | CEACAM5 |
| KCNN3 | XIST | SLCO1B3 |
| C9ORF65 | SORBS1 | SERPINB5 |
| PLUNC | CLPTM1 | PTPRH |
| TMEM45A | P4HA2 | SERPINB4 |
| LOC283177 | KRT17 | GSTT1 |
| GRP | ARHGEF10 | IL1RN |
| EFNB2 | HLF | ADM |
| FOXA2 | PCDHB5 | AKR1B10 |
| DCDC2 | SOX9 | ZBTB16 |
| WNK4 | PTGFR | SERPINB2 |
| FMN2 | QKI | CLCA1 |
| FXYD6 | CLEC7A | SDCBP2 |
| SLC13A2 | IPO11 | SCEL |
| SLC44A5 | OBSL1 | PHEX |
| WHSC2 | MS4A1 | AREG |
| ASCL1 | TNIK | SPRR3 |
| DUSP2 | HOXC4 | TCN1 |
| PCDH17 | LOC401074 | PRR4 |
| CDH2 | PRICKLE1 | FOSL1 |
| WNT5B | SPARCL1 | PHLDB2 |
| SFRP4 | CEP63 | PXDN |
| PEG10 | PPWD1 | KRT23 |
| STEAP4 | KLHL22 | HS6ST2 |
| GSTA2 | NR3C2 | TFCP2L1 |
| TMEM67 | DOC1 | DLG4 |
| KCNA1 | GEM | CTSL2 |
| TRIM55 | EMP1 | COPZ2 |
| RTN1 | EGR1 | SERPINB8 |
| ZNF667 | USP51 | CEL |
| SCN4B | PLD1 | SH3RF2 |
| CORO2B | SNF1LK2 | IL8RB |
| TCEAL2 | SLC5A1 | S100A10 |
| CHI3L1 | PCP4L1 | CA12 |
| DMXL2 | CAPNS2 | IL1R2 |
| TGM3 | TIMP3 | AADAC |
| GEM | GABBR1 | ARL4D |
| NAV3 | SLC6A6 | TRIM7 |
| EGR2 | FLJ41484 | PTGS2 |
| NPAS3 | CAV1 | HRASLS |
| SNF1LK2 | CEL | TOP2A |
| C8ORF42 | USP6 | SDPR |
| C6ORF201 | TDRD6 | TUBB6 |
| SLIT1 | LAIR1 | KRT13 |
| TDRD6 | ABCC4 | NGFRAP1L1 |
| NEB | PRDM16 | CLCNKB |
| FST | PART1 | S100A8 |
| IRS2 | FOSL1 | SRPX2 |
| BCAS3 | LSP1 | MX2 |
| LOC284412 | COL5A2 | C20ORF42 |
| DOC2A | EML5 | TNNI3 |
| SGCE | NPAS2 | C12ORF52 |
| BICC1 | GABRB3 | SLC6A8 |
| C14ORF132 | SLC13A4 | POSTN |
| PTPRM | CCR1 | ADAMTS9 |
| HIBADH | TNFAIP6 | DEFB1 |
| CDKL1 | FMNL2 | SLC7A11 |
| LSS | IREB2 | PKP2 |
| SCGB3A1 | ENPP2 | WDR72 |
| FOLR1 | RAD51L1 | EGR3 |
| FOSB | DAB2 | ARNTL2 |
| JAM3 | GPR137B | PMAIP1 |
| XIST | KLRB1 | TTLL12 |
| LRRIQ1 | RAET1E | C1ORF79 |
| HSPA12A | ASRGL1 | LOC157381 |
| RAI2 | ACE2 | PCOTH |
| ZNF663 | LYPLAL1 | CKAP4 |
| EGR1 | AP1S1 | HPSE |
| RASD1 | TSPAN5 | MTSS1 |
| ZNF418 | RSRC1 | TM4SF1 |
| MT1M | FKBP5 | FOSB |
| PER2 | DLG1 | FOXA3 |
| SLAMF7 | PRMT2 | SERPINE1 |
| SPP1 | SPRR3 | GMPR |
| SLC13A4 | MTHFD1L | CD93 |
| ZNF564 | ST8SIA1 | NTRK2 |
| TRPC6 | PTPN22 | FETUB |
| B4GALT1 | BCL2L1 | C17ORF28 |
| BRE | KIAA1904 | KRT24 |
| RAD51L1 | UBE3B | C12ORF54 |
| CHN2 | PMAIP1 | DQX1 |
| SLC24A5 | CCNB2 | ERBB3 |
| NID2 | KBTBD4 | GAS2L1 |
| IPO11 | JAK1 | CSF2RB |
| HRK | C1ORF51 | TLR4 |
| CXCL2 | GZMB | NP |
| GJA1 | BTBD9 | GCNT3 |
| PRKXP1 | C13ORF34 | MNT |
| CEP135 | PSMB7 | ATP10B |
| CXCL3 | PLK4 | S100A16 |
| ZNF331 | LAPTM4B | BFSP1 |
| ANXA6 | FLJ40432 | SULT2B1 |
| WDR60 | PLAC4 | FHL1 |
| GSTM5 | OAZ3 | PPARGC1A |
| PAPLN | ABHD2 | CCNA1 |
| FOS | DSC2 | LOC25845 |
| RNF190 | TRPC6 | GPRC5A |
| FRMD4A | TTTY15 | TRAF3IP3 |
| SCG2 | ABCA9 | UPK1B |
| CLASP2 | SERPINB8 | ANLN |
| CRIM1 | OSTBETA | TMEM75 |
| PRKCE | SAMD9 | LRRC8A |
| NR4A3 | CXCR6 | PCDHAC1 |
| CLGN | CDKN2B | FKBP1B |
| HHLA2 | ALPK3 | B3GNT6 |
| ERRFI1 | TFPI | FAM110C |
| FOXE1 | SMCHD1 | FAM46B |
| TMEM46 | CD36 | SFN |
| SCNN1G | P15RS | MKI67 |
| SYT13 | AADAC | CST1 |
| PRDM16 | SERPINB4 | KIAA1904 |
| SLC5A1 | SLC16A7 | CTNS |
| SLC1A1 | AGPAT4 | APOBEC3B |
| CYR61 | CALM1 | CBR3 |
| IGFBP5 | LARGE | SNCA |
| CCL18 | DUOX2 | MLZE |
| ITM2A | CXADR | EMP1 |
| LGR4 | CTTNBP2NL | FHL2 |
| ITPR1 | RNF150 | FCGR3B |
| KLF12 | TMPO | FBN2 |
| NPAS2 | CSF2RB | FLJ38773 |
| KCNK1 | EVI5 | KIAA1026 |
| AXUD1 | TBCA | CUGBP2 |
| CEL | LOC374443 | SLC25A25 |
| HOXC4 | LACTB | VRK3 |
| COL5A2 | UPK1B | MLKL |
| CNTD1 | HFE | DUSP3 |
| BIN3 | ANAPC5 | COBL |
| TNFAIP6 | WDR55 | GDF15 |
| FLCN | IGSF11 | PSTPIP2 |
| SLC7A7 | CDC20B | OSTBETA |
| ASB4 | SEC24A | KRT4 |
| C8ORF46 | CHAD | VPS37B |
| ODF1 | ZNF141 | EHD2 |
| SLC6A16 | TOP2A | KLHL18 |
| IRX1 | DLGAP1 | SYNJ2 |
| FAM51A1 | FAM83D | MFSD2 |
| CALCA | SEMA3C | EGR2 |
| EFHD1 | KLRC4 /// KLRK1 | MYL9 |
| PGAP1 | FBN2 | CD36 |
| TGIF | TANC2 | NMU |
| CYP2A13 | CREB5 | LY96 |
| JUNB | SFRS8 | CABYR |
| HIRA | MATN2 | HSPA6 |
| DPY19L2 | DPP4 | ITGAM |
| FLJ33360 | MAD2L1 | C1ORF53 |
| DNAJC4 | BFSP1 | ABCA1 |
| DKFZP686A01247 | SERPINE1 | HPGD |
| MYEF2 | CACNB4 | NEK2 |
| DEXI | ADM | UHRF1 |
| C6ORF153 | C11ORF9 | LMTK3 |
| KCNMB2 | NR2C2 | UBE2T |
| ISLR | SLC7A11 | TNNC1 |
| SLC5A9 | HRASLS | TXNL5 |
| C8ORF72 | SEC23IP | PLA2G4A |
| PCNX | GNG2 | CD44 |
| VPS37B | CPT1A | FPR1 |
| DNAJC5B | ZDHHC21 | FCAR |
| CCDC8 | GLS | SCG3 |
| WDR44 | GOPC | TTMA |
| ZNF382 | C15ORF48 | S100A9 |
| ABCC6 | BAX | CPT1A |
| SLC40A1 | SOX30 | HBEGF |
| MSR1 | HTR2B | SELL |
| P4HA2 | SDCBP2 | TMEM106C |
| GPR98 | GPR171 | EDIL3 |
| TLR8 | IRAK3 | DSG3 |
| BCL10 | CENPL | SCARNA2 |
| DMPK | NR1D2 | HK2 |
| MICAL3 | TSPAN2 | MLLT4 |
| TOX | CEACAM1 | LOC126917 |
| EFCAB1 | KLF4 | FAM83D |
| PIP | CFL2 | MNDA |
| ABP1 | GCNT3 | CCRN4L |
| C1ORF125 | MAWBP | DSCR1 |
| FBXL16 | ZNF396 | DPYSL3 |
| ZNF551 | C9ORF72 | GALE |
| SEC14L3 | OSTALPHA | ETV1 |
| GABRB3 | NRXN3 | CYR61 |
| GPR115 | LOC440895 | APBB1IP |
| PDPN | GZMH | SDF2L1 |
| ANKH | NAV1 | ASB16 |
| LOC286189 | TLE1 | SNORA68 |
| EHBP1L1 | KIF20A | FKBP11 |
| CST6 | BAG2 | ANXA1 |
| EHD4 | GON4L | MTP18 |
| ZBTB20 | MIPOL1 | ATF7 |
| CHN1 | TTC7B | SCNN1A |
| GBP1 | ST6GAL1 | IL1RL1 |
| MGC16703 | RIPK2 | CORO1C |
| KIAA1919 | LSM14B | LOC692247 |
| MGP | FAM80B | SAMD9 |
| KIAA1257 | DHX35 | PNKP |
| EPB41L2 | DNAJC12 | PLEKHJ1 |
| SERPINA6 | ITGB8 | TPX2 |
| LRP11 | NUSAP1 | ZNF57 |
| STS | UHRF1 | CREB5 |
| FLNB | PHLDB2 | SLC7A8 |
| CSMD1 | CD1C | BIRC5 |
| EGR3 | AKR1B10 | C15ORF48 |
| SLC4A4 | ZMYM5 | KLF9 |
| WDR17 | HOMER2 | DMRT2 |
| SLC1A2 | TM4SF1 | BCAT1 |
| ZNF292 | SLC39A8 | BAIAP2 |
| GPRASP1 | MATR3 | LOC389023 |
| SLC26A9 | NETO2 | CSNK1E |
| RPL15 | APOBEC3B | ITGA6 |
| TPM4 | IRF6 | TBX1 |
| SOCS3 | TBL1XR1 | LY6G5C |
| WNT2B | VPS35 | FAM101B |
| MAP3K12 | API5 | ITGA2B |
| ZNF19 | GZMA | IGFBPL1 |
| CD300LF | THRAP2 | RPP25 |
| CAMSAP1 | ABCC2 | GGCX |
| KIAA0232 | RASSF2 | SHMT2 |
| ZNF587 | CNTF | EIF4EBP2 |
| IL1B | C12ORF29 | PPP1R13L |
| MFAP3L | B3GNT6 | KCNK6 |
| CFDP1 | ARHGEF7 | LILRA2 |
| INSR | TCP11L2 | CREB3L4 |
| EIF1 | GPC1 | KRT8 |
| MYO15B | RHBDL2 | LGALS7 |
| ARHGEF12 | UBE2T | LOC440895 |
| C8ORF38 | SRPX2 | TPP2 |
| AQP9 | KCNE3 | C6ORF168 |
| PPWD1 | IL18RAP | STX6 |
| SKIL | ANLN | MGC14376 |
| ABCA9 | PXDN | FUT3 |
| DKFZP667F0711 | NEK2 | GAD1 |
| FNBP1 | CD109 | SPAG5 |
| LCMT2 | RAB5A | TMEPAI |
| RAB6B | ATP13A5 | CTPS |
| LOC692247 | B4GALT6 | TNFSF13B |
| PKD2 | NRP2 | FLAD1 |
| DAPK1 | PHACTR3 | NEK6 |
| LOC126917 | KIT | FBXL7 |
| RPH3AL | ATP10B | OSBP2 |
| ACADL | RRM2 | FPRL1 |
| DPYSL2 | DTNA | DGKA |
| GBP6 | FAM110C | CCDC40 |
| KLHL22 | UGT8 | ADAM21 |
| IFT88 | SCEL | FBXO31 |
| LIMS1 | MED31 | C12ORF57 |
| RPRM | RPL41 | ANKRD28 |
| CD163 | CD3G | SLCO2A1 |
| PPP1R9A | MUC12 | MICAL-L2 |
| RBP4 | STAT4 | ARNTL |
| NCALD | WDR72 | EGFL6 |
| CCNF | AGPS | PRSS33 |
| LUC7L2 | BCL2L14 | CCDC109B |
| ARMCX4 | MFSD2 | TFF1 |
| AQP1 | AKAP12 | MGC5590 |
| ARRB1 | GSN | RNF39 |
| NAG6 | NEK6 | SCO2 |
| MEIS2 | PLEKHG1 | UST |
| RAB3IP | CARS | NPAS2 |
| OXCT2 | AP1S3 | CROT |
| CHRNA9 | CD69 | PSCA |
| ZNF345 | CD1E | PHLDA1 |
| LOC221442 | ARNTL2 | LOC152225 |
| NELL2 | INPP4B | ITPR3 |
| PTGFR | SPECC1 | DHX35 |
| RASSF3 | SERPINB13 | BLM |
| TBX1 | EXOC6 | ZGPAT |
| CRISPLD2 | PTGS1 | TIMP3 |
| KCNJ1 | BDNF | NEDD9 |
| KIAA1909 | NMU | TMTC2 |
| LOC145837 | POSTN | RIPK2 |
| SEC14L5 | UEVLD | TACSTD2 |
| GADD45B | SAMSN1 | HOMER2 |
| CCDC40 | TPRXL | TLR8 |
| TREM1 | TCN1 | CLEC7A |
| LOC493754 | IL18R1 | LMNA |
| DPH1 | CD44 | SYTL4 |
| C8ORF34 | PRR4 | EFHD2 |
| ARID5B | CEACAM5 | PTRF |
| ATF7 | SHH | PON3 |
| HERC5 | SLCO1B3 | DDIT3 |
| CXCR7 | SLC24A3 | PDK4 |
| LOC200609 | IGF2BP3 | MND1 |
| SCUBE3 | SERPINB10 | HES2 |
| IDS | DPYSL3 | APEX2 |
| SUV420H1 | CLCA1 | SNHG3 |
| ETV3 | CST1 | CECR5 |
| TYSND1 | CD200R1 | PLCB1 |
| CFD | PTPRH | GNL1 |
| COQ9 | GATA2 | SLC39A14 |
| GRM5 | CPA3 | RUSC1 |
| GAS1 | NTRK2 | PRKAB1 |
| DPYD | MS4A2 | TBC1D7 |
| RAPGEF6 | SERPINB2 | SOX7 |
| IFI6 | | AP1S3 |
| CYP2D6 | | SRD5A1 |
| TWIST1 | | TBC1D16 |
| NR1I2 | | VPS26A |
| SERPING1 | | WDR55 |
| RBMS3 | | FOS |
| PBX1 | | SERPINB10 |
| PILRA | | CSTA |
| SLC9A11 | | NOL5A |
| SIAH1 | | SIRPB1 |
| PISD | | HN1 |
| SOX9 | | DSC2 |
| RAPH1 | | STX10 |
| FOSL1 | | ZCCHC10 |
| FCGR3B | | SEC14L1 |
| ZNF529 | | GGH |
| CATSPER2 | | PTAFR |
| SUZ12 | | TIGD3 |
| TACR1 | | COX10 |
| SLC16A6 | | STARD13 |
| LRP1 | | PUSL1 |
| MYO1E | | LOC401074 |
| JUN | | ADAMTSL4 |
| CFLAR | | DOHH |
| OGFOD1 | | TMEM64 |
| AK2 | | PLK4 |
| WFDC3 | | PTP4A1 |
| CLEC4E | | RRM2 |
| ARHGEF7 | | ME1 |
| MAP2 | | C14ORF147 |
| PPOX | | KBTBD2 |
| SNED1 | | NUFIP2 |
| FBXL7 | | TSTA3 |
| SEC14L4 | | FA2H |
| COX11 | | CLTB |
| CYBB | | MGC16275 |
| BTBD11 | | WDR4 |
| DKK3 | | C22ORF28 |
| MSLN | | KIAA1754 |
| SLCO3A1 | | CDR1 |
| NFIA | | MUC6 |
| PDE5A | | CD300A |
| KRT17 | | RASSF2 |
| INTS10 | | FAM3B |
| CKMT2 | | DNAJC4 |
| PAX1 | | FGR |
| LOC157381 | | PLEKHG1 |
| MPP7 | | RUNX2 |
| IFI44L | | LOC154761 |
| PCOLCE2 | | DTNA |
| LOC644192 | | SOX5 |
| SFRS8 | | TREX1 |
| IL1R2 | | KIF19 |
| KBTBD4 | | PHF23 |
| MYL9 | | PPP1R16B |
| HEBP1 | | EMR2 |
| CORO1C | | TIMP1 |
| PLAT | | TNS4 |
| CD8A | | C17ORF67 |
| DOK1 | | LSS |
| TFAP2C | | FBXO6 |
| TSPAN5 | | GMDS |
| LY6D | | HSPC111 |
| TANC2 | | SLC25A20 |
| MIPOL1 | | HRK |
| CD24 | | DOPEY2 |
| CPT1B | | C15ORF37 |
| ABCA1 | | MGAT4B |
| TSHZ2 | | MUC5AC |
| RHBDL2 | | UBE3B |
| PELO | | UACA |
| MYOT | | GPX2 |
| PHACTR3 | | SPSB1 |
| CACNB4 | | E2F2 |
| SLC16A1 | | SNORD8 |
| NRIP3 | | MSLN |
| DEFB1 | | CCDC80 |
| UPK1B | | SFRP4 |
| DUOX2 | | CKS2 |
| RPL41 | | CYP39A1 |
| RAB15 | | GSN |
| SOS2 | | ESPN |
| CAPN13 | | PFKFB2 |
| SPINK5 | | IFT81 |
| IL17RB | | F5 |
| UHRF1 | | PALLD |
| HOXB3 | | IKIP |
| EMR2 | | TMEM167 |
| GON4L | | PTPRE |
| MGAT4A | | STATH |
| CD109 | | STRA6 |
| JAK1 | | CNTF |
| KRT13 | | SIRPA |
| PRR5 | | CCBP2 |
| COPZ2 | | P2RY1 |
| TIA1 | | FAM111B |
| UBE2T | | LACTB |
| CD247 | | SLC9A3 |
| SERPINB9 | | SAMHD1 |
| CA12 | | BAG5 |
| SEC24A | | UCK2 |
| CALM1 | | TNC |
| DQX1 | | AQP9 |
| GSTM3 | | TJP1 |
| CDC20B | | KDELR3 |
| CXCL14 | | GALNT5 |
| CENPL | | ZMYND19 |
| BMP7 | | PHKA1 |
| FAM101B | | SEC14L5 |
| MUC5AC | | SNAPC4 |
| MAPKAP1 | | CLCA4 |
| TTC7B | | IRF7 |
| GVIN1 | | BCL2L14 |
| C11ORF9 | | TPM4 |
| FLJ40432 | | HSPC159 |
| DAPK2 | | HBD |
| TLK1 | | HAS3 |
| KIF20A | | NCF4 |
| GFI1 | | MED25 |
| CXCR6 | | PLAT |
| FLI1 | | MLYCD |
| DPYSL3 | | CNIH3 |
| MTHFD1L | | ZNF155 |
| GPR68 | | CHI3L1 |
| DGKA | | PROM2 |
| PSPC1 | | PIGQ |
| SRPX2 | | TFEB |
| ADRA2A | | MPP1 |
| APOBEC3B | | LOC286297 |
| RNF43 | | HMG20B |
| B3GALT5 | | HYAL1 |
| C15ORF48 | | FLI1 |
| MRPL19 | | KCNE3 |
| IRAK3 | | CNTNAP3B |
| CALD1 | | SYNGR1 |
| C15ORF34 | | MAP3K12 |
| TMEM64 | | KCTD14 |
| CORO1A | | GRM6 |
| MATR3 | | PTK6 |
| DLG1 | | DOK3 |
| DOCK10 | | CDK2AP2 |
| LMBRD2 | | GCAT |
| SEC23IP | | LOC151534 |
| MLKL | | SOCS3 |
| SLC7A11 | | DOK1 |
| FAM83D | | IL18R1 |
| LOC25845 | | GPT2 |
| AADAC | | LIN7A |
| HRASLS | | BMP2 |
| BAG2 | | B3GALT6 |
| SLC39A8 | | CLEC4E |
| PSMB7 | | GFER |
| HPCAL1 | | DBN1 |
| SOX30 | | SPP1 |
| CCNB2 | | SLC25A39 |
| GZMB | | PAQR6 |
| SMCHD1 | | FBLIM1 |
| EPAS1 | | C200RF102 |
| SDCBP2 | | PCDHB14 |
| DNASE1L3 | | KIF20A |
| AKR1B10 | | ST14 |
| FBN2 | | YEATS2 |
| PHLDB2 | | MXD1 |
| MALT1 | | AURKA |
| VSIG2 | | LSP1 |
| CD44 | | C12ORF5 |
| TOP2A | | CYP2D6 |
| CD3D | | RPH3AL |
| MMP28 | | C9ORF66 |
| KLF4 | | TWIST1 |
| CARS | | NR4A3 |
| MED31 | | ECT2 |
| NAV1 | | SLA |
| ARHGDIB | | CA1 |
| SERPINB8 | | TPMT |
| TBL1Y | | DNM3 |
| RRM2 | | RPS6KA1 |
| SNCA | | IGSF11 |
| SH3RF2 | | CDC20B |
| FKBP1B | | EPAG |
| CEACAM1 | | HCLS1 |
| BCMO1 | | CD274 |
| ANLN | | CST6 |
| CD48 | | C21ORF129 |
| SHH | | PPAN |
| GPR171 | | FKBP14 |
| CSF2RB | | C9ORF152 |
| PCOTH | | MAPK8IP3 |
| TTTY15 | | PHTF1 |
| SPECC1 | | TACC1 |
| KCNE3 | | BCL2A1 |
| UEVLD | | AGPS |
| RIPK2 | | AHSG |
| PTPN22 | | AYTL2 |
| TM4SF1 | | LOC89944 |
| SCML4 | | DAXX |
| HYAL1 | | GABRB3 |
| DDX3Y | | MTG1 |
| CTTNBP2NL | | STS |
| UBE3B | | CDC25A |
| PLA2G4A | | CHAF1A |
| UTY | | CYP2R1 |
| SERPINB4 | | SNORA71B |
| AGPS | | JUN |
| B3GNT6 | | TBC1D2B |
| MFSD2 | | USP18 |
| GAD1 | | ALS2CL |
| ZFY | | PLA2G7 |
| PXDN | | DFNA5 |
| ATP10B | | ZNF134 |
| FAM43A | | PPARD |
| C12ORF29 | | HIST1H2BM |
| INPP4B | | MARVELD3 |
| CDKN2B | | GLRX |
| C6ORF168 | | H2AFX |
| KLRC4 /// KLRK1 | | DAPK2 |
| TRAF3IP3 | | KIAA0391 |
| IGF2BP3 | | IGSF6 |
| PLK4 | | BBOX1 |
| TMEM117 | | BTNL9 |
| WDR72 | | TMEM43 |
| NEK6 | | VARS |
| PLEKHA2 | | DPH1 |
| NETO2 | | GPS2 |
| RBMS1 | | CBR1 |
| C1ORF51 | | RFFL |
| CMYA5 | | TAT |
| LOC374443 | | TNFRSF10C |
| GZMA | | TMED9 |
| KLRB1 | | ENDOGL1 |
| EXOC6 | | HYLS1 |
| SAMD4A | | NRIP3 |
| B4GALT6 | | LATS2 |
| CD36 | | DBNL |
| GPR137B | | DDX39 |
| RAB5A | | DCBLD1 |
| SPRR3 | | AP2S1 |
| CHAD | | COQ3 |
| FAM110C | | CD163 |
| CD69 | | BACH1 |
| STATH | | LOC399900 |
| AP1S3 | | RPS27L |
| TBC1D16 | | ITGB6 |
| MATN2 | | KBTBD4 |
| PLEKHG1 | | PSPH |
| BCL2L14 | | AXUD1 |
| GJC1 | | KIAA1212 |
| STAT4 | | LOC153684 |
| SLCO1B3 | | ZNF317 |
| SERPINB13 | | SLCO3A1 |
| HOMER2 | | CENPN |
| IL18RAP | | BCR |
| LAPTM4B | | MESDC1 |
| TPRXL | | PNPLA4 |
| TLE1 | | TP53I3 |
| GALNT10 | | C8ORF4 |
| ASRGL1 | | CXCL9 |
| FOXA3 | | NLN |
| SCEL | | GADD45B |
| GNG2 | | TSPAN2 |
| NMU | | C19ORF59 |
| GSN | | TMEM2 |
| DHX35 | | CSGLCA-T |
| RASSF2 | | NAV1 |
| TFF1 | | ANXA2 |
| GPC1 | | TRIM35 |
| GCNT3 | | GMFG |
| ATP13A5 | | RAP2B |
| PTPRH | | KIAA0101 |
| HMGCS2 | | ZFP36 |
| CD3G | | FKSG24 |
| TCN1 | | DAAM1 |
| KIT | | BAP1 |
| PTGS1 | | CSPG2 |
| SAMSN1 | | ZWINT |
| ARNTL2 | | AKR7A2 |
| CD1E | | LOC152485 |
| POSTN | | TAGAP |
| CD1C | | LY6D |
| SLC24A3 | | HERC5 |
| PRR4 | | FAM83F |
| AKAP12 | | CCNB1 |
| CEACAM5 | | ZNF165 |
| NTRK2 | | EVI2B |
| SERPINB2 | | HSPA5 |
| CST1 | | TMEM118 |
| SERPINB10 | | ZDHHC4 |
| CD200R1 | | KRT7 |
| CLCA1 | | CCBL1 |
| IL18R1 | | MYC |
| GATA2 | | YKT6 |
| MS4A2 | | ISG20L2 |
| CPA3 | | CRIP2 |
| | | LRRC43 |
| | | SLC45A4 |
| | | CDCP1 |
| | | ZNF592 |
| | | MFN1 |
| | | ERRFI1 |
| | | ABHD12 |
| | | TUBB4 |
| | | NADK |
| | | GALK1 |
| | | WWP2 |
| | | POLRMT |
| | | SLA2 |
| | | IGF2BP3 |
| | | TFAP2C |
| | | SLC25A13 |
| | | TANC2 |
| | | KRT6B |
| | | AP1M1 |
| | | FBP1 |
| | | LOC648987 |
| | | CDC20 |
| | | CDH26 |
| | | IL18RAP |
| | | TFPI2 |
| | | INPP4B |
| | | YRDC |
| | | CXCL10 |
| | | CENPF |
| | | OSTALPHA |
| | | TNFRSF13B |
| | | SEPX1 |
| | | SERPINB13 |
| | | MICB |
| | | CAV1 |
| | | MATN2 |
| | | ACSS1 |
| | | TGIF |
| | | BPIL1 |
| | | NEU1 |
| | | DYRK3 |
| | | TDP1 |
| | | CLPTM1 |
| | | NFKBIA |
| | | RPL28 |
| | | SFXN4 |
| | | FBXO32 |
| | | ANKRD9 |
| | | KLHL9 |
| | | CFL2 |
| | | TTC30A |
| | | PLXNB1 |
| | | FAM83B |
| | | ABO |
| | | RGS2 |
| | | SAMD4A |
| | | GALNTL4 |
| | | SLC1A3 |
| | | RGS14 |
| | | ICAM3 |
| | | KIT |
| | | SNORA74A |
| | | EMB |
| | | QKI |
| | | TMEM117 |
| | | PPP1R15A |
| | | ATF3 |
| | | C15ORF34 |
| | | CENPL |
| | | IGFBP3 |
| | | IRX1 |
| | | TMED4 |
| | | IDS |
| | | DUOXA1 |
| | | SOCS1 |
| | | ETHE1 |
| | | COMMD5 |
| | | CDKN3 |
| | | PPP1R16A |
| | | GZMB |
| | | TGFBI |
| | | SVEP1 |
| | | UCHL1 |
| | | LOC90379 |
| | | KIAA1257 |
| | | SMAP1L |
| | | PACSIN2 |
| | | LOXL4 |
| | | CCND3 |
| | | TSSC1 |
| | | C3ORF62 |
| | | RTP4 |
| | | EDNRB |
| | | OSM |
| | | C13ORF34 |
| | | CCL2 |
| | | KLF6 |
| | | LTB4R |
| | | EHD4 |
| | | RCCD1 |
| | | PLK3 |
| | | TRPC6 |
| | | GBP3 |
| | | KIAA0241 |
| | | BDNF |
| | | SQLE |
| | | FOLH1 |
| | | RECQL4 |
| | | SLC39A11 |
| | | HPCAL1 |
| | | SLC7A6 |
| | | CAMSAP1 |
| | | GJB3 |
| | | PCP4L1 |
| | | ZNF211 |
| | | DDEF1 |
| | | STOX2 |
| | | FAM51A1 |
| | | CCR1 |
| | | CDK5 |
| | | CD200R1 |
| | | VPS72 |
| | | C6ORF115 |
| | | SENP8 |
| | | HS3ST1 |
| | | ACP6 |
| | | C19ORF21 |
| | | SLC24A3 |
| | | WNT6 |
| | | OGFOD1 |
| | | PHLPPL |
| | | MRVI1 |
| | | SIN3B |
| | | GGA2 |
| | | HSPG2 |
| | | C3ORF30 |
| | | BIRC3 |
| | | PYGB |
| | | SYN2 |
| | | CEP250 |
| | | PGM2 |
| | | AGPAT7 |
| | | BIN2 |
| | | TNFAIP6 |
| | | VWF |
| | | GUK1 |
| | | LOC81691 |
| | | CEACAM1 |
| | | DIAPH2 |
| | | TTC15 |
| | | TSHZ3 |
| | | RPL41 |
| | | NOL3 |
| | | FNIP1 |
| | | CARS |
| | | C19ORF54 |
| | | LRP11 |
| | | TUBG1 |
| | | ARHGEF10 |
| | | WDR5 |
| | | TMEM22 |
| | | C12ORF53 |
| | | FAT2 |
| | | ACOX3 |
| | | PPP4C |
| | | NPL |
| | | NOP17 |
| | | C11ORF2 |
| | | MS4A2 |
| | | BSDC1 |
| | | C18ORF8 |
| | | PILRB |
| | | SH3D19 |
| | | ALG14 |
| | | PRKAB2 |
| | | PTPDC1 |
| | | FGD5 |
| | | TRIMP1 |
| | | SIGLECP16 |
| | | NDST1 |
| | | NUSAP1 |
| | | SLC39A8 |
| | | SURF6 |
| | | KLF4 |
| | | MVP |
| | | SERPINB9 |
| | | CSRP2BP |
| | | PELI1 |
| | | C14ORF112 |
| | | HIP1 |
| | | CCDC37 |
| | | MCL1 |
| | | KRT17 |
| | | GPR172A |
| | | SIDT1 |
| | | PGLS |
| | | IER2 |
| | | ATP13A5 |
| | | IRF8 |
| | | FAM79B |
| | | MAP3K9 |
| | | SEC22C |
| | | FGF13 |
| | | ACOT11 |
| | | UNC45A |
| | | DPY19L1 |
| | | MGC50722 |
| | | MYD88 |
| | | TFPT |
| | | CDR2L |
| | | DDX58 |
| | | EML2 |
| | | DNAPTP6 |
| | | C3ORF14 |
| | | ATP6V1G3 |
| | | WDR45 |
| | | CYB5R2 |
| | | CDKL3 |
| | | RAB40C |
| | | RASD1 |
| | | RGS10 |
| | | ARHGDIB |
| | | ANXA3 |
| | | LOC401093 |
| | | TLE1 |
| | | NUDT1 |
| | | PDIA5 |
| | | GRM7 |
| | | DOK2 |
| | | MID2 |
| | | SRM |
| | | BMP7 |
| | | ARHGAP25 |
| | | OBFC2A |
| | | DEFB4 |
| | | SORBS2 |
| | | COX15 |
| | | CNNM3 |
| | | PERP |
| | | FAM83C |
| | | RAB35 |
| | | YY2 |
| | | CALD1 |
| | | SNAI2 |
| | | LAYN |
| | | LOC654433 |
| | | C1ORF74 |
| | | LZTR1 |
| | | MSN |
| | | CHRNE |
| | | IRF6 |
| | | VPS41 |
| | | COCH |
| | | ARHGAP17 |
| | | ADAM19 |
| | | FCGR2A |
| | | CR1 |
| | | TMEM136 |
| | | KCNK5 |
| | | TPK1 |
| | | CTF1 |
| | | SLC25A35 |
| | | GLYATL2 |
| | | GDA |
| | | PRKAR1B |
| | | ALK |
| | | PROCR |
| | | DLX4 |
| | | SOX9 |
| | | INTS1 |
| | | TRAF1 |
| | | RHBDL2 |
| | | TRAPPC2L |
| | | SYK |
| | | FAM71A |
| | | NOL6 |
| | | GPSM3 |
| | | PPARG |
| | | PDE4D |
| | | ZBTB47 |
| | | GPR56 |
| | | CYBB |
| | | D4S234E |
| | | BRE |
| | | MARK4 |
| | | MYO15B |
| | | DUOX2 |
| | | BAG1 |
| | | PDE3A |
| | | C21ORF108 |
| | | IL1B |
| | | RNASE2 |
| | | KIAA1949 |
| | | DAB2 |
| | | LPL |
| | | TTLL10 |
| | | AGPAT4 |
| | | PLXDC2 |
| | | C17ORF62 |
| | | ZNF593 |
| | | ASB10 |
| | | SLC7A7 |
| | | GSPT2 |
| | | TCF2 |
| | | MMAA |
| | | PTPN23 |
| | | KIF4A |
| | | EGR1 |
| | | SLC28A3 |
| | | BID |
| | | DDEF2 |
| | | C8ORF60 |
| | | CPE |
| | | DMPK |
| | | USP45 |
| | | LAIR1 |
| | | PRG1 |
| | | GATA2 |
| | | CLIC5 |
| | | COP1 |
| | | SLC16A1 |
| | | ANKRD25 |
| | | JUNB |
| | | MGC12916 |
| | | PAX8 |
| | | MXRA5 |
| | | GJC1 |
| | | P2RY13 |
| | | C12ORF61 |
| | | MAFB |
| | | VTCN1 |
| | | ALDH3A1 |
| | | NUFIP1 |
| | | PYCARD |
| | | CLEC4A |
| | | SEC61A1 |
| | | BAD |
| | | METTL9 |
| | | ASRGL1 |
| | | MRPS15 |
| | | ZNF561 |
| | | MEST |
| | | TAOK2 |
| | | ARS2 |
| | | CARM1 |
| | | MTX1 |
| | | LIMK1 |
| | | VAMP5 |
| | | C1ORF100 |
| | | GRIK2 |
| | | C4ORF9 |
| | | PXMP4 |
| | | MRPL41 |
| | | SLC4A1 |
| | | CDC34 |
| | | DLG1 |
| | | HCG4 |
| | | KRT5 |
| | | MASA |
| | | DDX54 |
| | | ADH4 |
| | | RABAC1 |
| | | R3HCC1 |
| | | PGD |
| | | USP36 |
| | | LIMS1 |
| | | FECH |
| | | METT5D1 |
| | | APOA1BP |
| | | MRPL44 |
| | | CYB5B |
| | | GAPDH |
| | | GIMAP4 |
| | | SERPINA1 |
| | | MLXIP |
| | | DUSP1 |
| | | NRF1 |
| | | VMD2 |
| | | AKT1S1 |
| | | KIAA0182 |
| | | CAMKK2 |
| | | LOC284889 |
| | | TTC21A |
| | | KIAA0701 |
| | | MRPL4 |
| | | CD63 |
| | | TMEM131 |
| | | CXORF26 |
| | | HDAC8 |
| | | NCALD |
| | | SLC23A3 |
| | | XLKD1 |
| | | EMILIN2 |
| | | TREM1 |
| | | CDRT1 |
| | | NDUFS8 |
| | | TRERF1 |
| | | PQBP1 |
| | | FMO3 |
| | | ALDH1L1 |
| | | NRP2 |
| | | TSPAN5 |
| | | C3ORF64 |
| | | RFXANK |
| | | FAM111A |
| | | GPR146 |
| | | ZBTB3 |
| | | IL18BP |
| | | HTR2B |
| | | FLJ90757 |
| | | BCL7A |
| | | C10ORF47 |
| | | WAS |
| | | HIST1H4L |
| | | ZNF569 |
| | | FOXO3A |
| | | DOC2A |
| | | E2F7 |
| | | CPM |
| | | SERINC2 |
| | | F2RL1 |
| | | THOC4 |
| | | RAET1E |
| | | RPRC1 |
| | | CCL24 |
| | | MYO1E |
| | | ALKBH4 |
| | | TAC4 |
| | | DTNBP1 |
| | | IPO4 |
| | | HLA-DRB4 |
| | | GAS2L3 |
| | | UQCRC1 |
| | | CACNB4 |
| | | ST6GAL1 |
| | | C11ORF9 |
| | | MCC |
| | | PSMD3 |
| | | AQP1 |
| | | FBXW12 |
| | | YARS |
| | | SHROOM3 |
| | | ACSL1 |
| | | KCTD3 |
| | | BNC1 |
| | | LASS6 |
| | | GPX1 |
| | | FAM43A |
| | | MAN2C1 |
| | | PAPOLG |
| | | ORAOV1 |
| | | FAM62C |
| | | RNASEH1 |
| | | FLJ40432 |
| | | ELN |
| | | GSK3A |
| | | TSPO |
| | | PRRT3 |
| | | FAM126A |
| | | WDSUB1 |
| | | CNFN |
| | | ABCC2 |
| | | AQP5 |
| | | TLR6 |
| | | HS3ST3B1 |
| | | SLC2A1 |
| | | N-PAC |
| | | DUSP19 |
| | | ZC3H12A |
| | | RPS6KA3 |
| | | C16ORF7 |
| | | SPINK5 |
| | | LIPA |
| | | PPM1F |
| | | PRR8 |
| | | EPB41 |
| | | PICK1 |
| | | SDC4 |
| | | EIF4EBP1 |
| | | SLC5A9 |
| | | HNRPM |
| | | VSIG2 |
| | | GEMIN4 |
| | | PVT1 |
| | | CCL18 |
| | | BTK |
| | | BAT3 |
| | | TNFAIP8L3 |
| | | SERTAD3 |
| | | CASKIN1 |
| | | CCNA2 |
| | | ARL15 |
| | | GIPR |
| | | TGFB2 |
| | | CAMP |
| | | TNKS1BP1 |
| | | GPR65 |
| | | NOS3 |
| | | IL17RB |
| | | RNF43 |
| | | HNRPA2B1 |
| | | HMOX2 |
| | | SNX12 |
| | | PLSCR1 |
| | | DNAL4 |
| | | EME2 |
| | | GJB5 |
| | | DAB2IP |
| | | JOSD1 |
| | | PRAF2 |
| | | PIN1 |
| | | RAB37 |
| | | C19ORF46 |
| | | NLGN2 |
| | | SLC31A2 |
| | | UQCRB |
| | | MMP28 |
| | | PTGS1 |
| | | CHEK1 |
| | | PPOX |
| | | COQ9 |
| | | MYH9 |
| | | FAM24B |
| | | TCEB3C |
| | | C16ORF58 |
| | | ARFRP1 |
| | | FRAT2 |
| | | TACC2 |
| | | CYP2C18 |
| | | CCT5 |
| | | DIAPH1 |
| | | CPA3 |
| | | MT1M |
| | | FCER1G |
| | | MAZ |
| | | ARID5B |
| | | ST7 |
| | | GCC1 |
| | | FLJ10241 |
| | | KLHL6 |
| | | ADRBK2 |
| | | KALRN |
| | | LOC390940 |
| | | PLOD2 |
| | | CD48 |
| | | CLEC12A |
| | | MMD |
| | | DIP2A |
| | | PDAP1 |
| | | ARIH2 |
| | | RPL22 |
| | | PFAS |
| | | XRCC5 |
| | | SCHIP1 |
| | | CTHRC1 |
| | | BCAS3 |
| | | XK |
| | | TXNRD3 |
| | | WFDC3 |
| | | DOCK8 |
| | | LOC202051 |
| | | BNIP3 |
| | | NCAPH2 |
| | | LOC285033 |
| | | ADH1B |
| | | RNF13 |
| | | FAM98C |
| | | NETO2 |
| | | TAGLN2 |
| | | DOM3Z |
| | | IRS1 |
| | | SLC35A2 |
| | | B3GALTL |
| | | HMHA1 |
| | | CRAT |
| | | GOLGA2 |
| | | EDEM2 |
| | | MBNL2 |
| | | MCART1 |
| | | PIGG |
| | | SEC23IP |
| | | FAIM3 |
| | | VWCE |
| | | LOC440896 |
| | | DLGAP1 |
| | | CAPN2 |
| | | MAPK13 |
| | | SEC24D |
| | | LHX4 |
| | | MTHFD1L |
| | | ZNF438 |
| | | GON4L |
| | | MRPL53 |
| | | SPECC1 |
| | | KIAA1920 |
| | | HMGCS2 |
| | | C9ORF3 |
| | | THBS1 |
| | | DRD2 |
| | | ZNF415 |
| | | TM6SF1 |
| | | AZGP1 |
| | | C1ORF96 |
| | | USP46 |
| | | MINK1 |
| | | SYNGR2 |
| | | TRAM2 |
| | | LRRC8C |
| | | NFATC2IP |
| | | CD5L |
| | | LSM4 |
| | | PIK3R1 |
| | | DFNB31 |
| | | TCF7L2 |
| | | PVRL1 |
| | | GYPC |
| | | HEMGN |
| | | ZYX |
| | | PRKACA |
| | | RNF150 |
| | | LARGE |
| | | GTF3C5 |
| | | LOC284385 |
| | | LEF1 |
| | | CCDC94 |
| | | SMPD2 |
| | | HSPBP1 |
| | | PTPRO |
| | | IQCG |
| | | PCOLCE2 |
| | | TBC1D1 |
| | | TCP11L2 |
| | | MCM6 |
| | | CDC2 |
| | | CLMN |
| | | SPOCD1 |
| | | DDX49 |
| | | OASL |
| | | SPATA13 |
| | | IPPK |
| | | FAM120A |
| | | C1ORF204 |
| | | C16ORF57 |
| | | LAPTM4B |
| | | BRD3 |
| | | SLC9A2 |
| | | MRPL12 |
| | | CD300LF |
| | | ABL2 |
| | | VAMP4 |
| | | LRRC36 |
| | | E2F6 |
| | | CDKL1 |
| | | GSTM3 |
| | | RPL10A |
| | | MME |
| | | CFLAR |
| | | ST8SIA1 |
| | | CUL7 |
| | | SLC26A2 |
| | | ACTN4 |
| | | SRPRB |
| | | DHRS13 |
| | | MFI2 |
| | | ELOVL6 |
| | | UPP1 |
| | | NUP188 |
| | | RSRC1 |
| | | PUS1 |
| | | ATP1A1 |
| | | EFCAB1 |
| | | ENTPD4 |
| | | POLE3 |
| | | FLJ41484 |
| | | PCSK6 |
| | | TREML4 |
| | | KLK12 |
| | | EVI5 |
| | | SIAE |
| | | LRRC54 |
| | | TMEM86B |
| | | ARAF |
| | | RUNX1 |
| | | PTTG2 |
| | | CLCN5 |
| | | BTBD11 |
| | | AP4S1 |
| | | ATP12A |
| | | GDAP1 |
| | | ARHGAP23 |
| | | IL15RA |
| | | SLC16A7 |
| | | NGB |
| | | MAST2 |
| | | SMAP1 |
| | | BCL10 |
| | | EMR3 |
| | | NCF2 |
| | | FBLN1 |
| | | FAM107B |
| | | MX1 |
| | | ZBTB43 |
| | | NCAPD2 |
| | | VPS52 |
| | | TOM1 |
| | | DYNLL1 |
| | | AP4E1 |
| | | UGT8 |
| | | LPP |
| | | C12ORF48 |
| | | CD53 |
| | | OAZ3 |
| | | CMYA5 |
| | | PSMD9 |
| | | MKL1 |
| | | SAMSN1 |
| | | MOBKL2C |
| | | PEX11A |
| | | FAM89A |
| | | PILRA |
| | | ZFYVE9 |
| | | FAM83A |
| | | ABHD2 |
| | | SGSH |
| | | RBMS3 |
| | | TFE3 |
| | | AACS |
| | | SLC5A6 |
| | | ANKMY1 |
| | | FAM107A |
| | | PPP1R7 |
| | | ABCC10 |
| | | PLAUR |
| | | ALOX5 |
| | | KLF8 |
| | | FOXD1 |
| | | RHOC |
| | | EPSTI1 |
| | | KSR1 |
| | | PLAC4 |
| | | CAPZA2 |
| | | SOS2 |
| | | TSC22D1 |
| | | LSM7 |
| | | CHAD |
| | | MAPKAP1 |
| | | STK24 |
| | | ODZ4 |
| | | NRG2 |
| | | SELPLG |
| | | RASIP1 |
| | | AKAP12 |
| | | TYR |
| | | TMED3 |
| | | TFPI |
| | | PABPC1 |
| | | ZCCHC17 |
| | | GSR |
| | | ETV3 |
| | | FOXK2 |
| | | GALNT10 |
| | | BCL2L1 |
| | | SPON2 |
| | | MCOLN1 |
| | | TRAK1 |
| | | SELI |
| | | NT5DC2 |
| | | ALAS1 |
| | | ELK1 |
| | | B4GALT3 |
| | | MRPL37 |
| | | ARHGEF2 |
| | | GTF3C1 |
| | | HEY2 |
| | | RPS24 |
| | | IGFBP2 |
| | | MGMT |
| | | N4BP1 |
| | | TRPS1 |
| | | HIRA |
| | | MFSD1 |
| | | PRDM2 |
| | | DNAJC8 |
| | | DSC3 |
| | | VKORC1L1 |
| | | TOB2 |
| | | NBL1 |
| | | MAMDC2 |
| | | PNPLA3 |
| | | OAS1 |
| | | PSMB2 |
| | | SEMA4A |
| | | CNDP2 |
| | | EIF3S4 |
| | | ADH6 |
| | | RBP4 |
| | | ZNF778 |
| | | TLR1 |
| | | AIM2 |
| | | TNNT3 |
| | | ALPK3 |
| | | BTBD9 |
| | | SLC18A2 |
| | | NUMBL |
| | | NFIA |
| | | USP33 |
| | | DKFZP564C152 |
| | | MEF2C |
| | | ZBTB25 |
| | | PER2 |
| | | MLLT6 |
| | | DUSP2 |
| | | TMEM176B |
| | | PSMA5 |
| | | RTTN |
| | | FBXW10 |
| | | FBXL17 |
| | | METTL6 |
| | | ERBB2 |
| | | TTC5 |
| | | TMEM106A |
| | | BIRC1 |
| | | SUV420H1 |
| | | SKIL |
| | | TNS1 |
| | | PLEKHA2 |
| | | FBXO3 |
| | | POR |
| | | MYEF2 |
| | | HNRPA0 |
| | | MYOT |
| | | ARL3 |
| | | KIAA0232 |
| | | RAB28 |
| | | IREB2 |
| | | SCNN1B |
| | | CSTF2T |
| | | ARHGAP1 |
| | | GOPC |
| | | KCTD15 |
| | | ITGAE |
| | | STAT6 |
| | | HERC6 |
| | | RND2 |
| | | MXRA7 |
| | | PIK4CB |
| | | SLC24A5 |
| | | CDK2 |
| | | DISC1 |
| | | TEX9 |
| | | NHS |
| | | CYP17A1 |
| | | SIRT3 |
| | | ARIH1 |
| | | USP1 |
| | | NCOA7 |
| | | RRAD |
| | | LMO7 |
| | | TMSB4Y |
| | | ZNF704 |
| | | DDX3Y |
| | | GBP4 |
| | | RALGPS1 |
| | | ZHX2 |
| | | C12ORF22 |
| | | GRB2 |
| | | SFXN2 |
| | | TNKS |
| | | NRIP2 |
| | | FLNB |
| | | ARRB1 |
| | | FSTL1 |
| | | FBXL16 |
| | | CACHD1 |
| | | SDAD1 |
| | | PTPRJ |
| | | IBSP |
| | | ZNF667 |
| | | BPGM |
| | | ID4 |
| | | NDE1 |
| | | XYLB |
| | | RASSF3 |
| | | EXOC7 |
| | | GEM |
| | | RAG1 |
| | | PPFIBP1 |
| | | ZMYM2 |
| | | BNC2 |
| | | SUPT6H |
| | | IQWD1 |
| | | CLN8 |
| | | SYNPR |
| | | ROBO1 |
| | | CGNL1 |
| | | FARP2 |
| | | MRPS22 |
| | | OGDH |
| | | CYP2C9 |
| | | SRC |
| | | BTRC |
| | | EIF2C4 |
| | | KCNK1 |
| | | TNPO1 |
| | | TAF15 |
| | | SASH1 |
| | | LYNX1 |
| | | RANBP17 |
| | | COG1 |
| | | IL1A |
| | | OTUD7B |
| | | PRICKLE1 |
| | | ANTXR1 |
| | | TMEM87A |
| | | TLK1 |
| | | PHKG2 |
| | | DSCAML1 |
| | | RAVER2 |
| | | FANCB |
| | | TMEM67 |
| | | SEMA3C |
| | | ZNF7 |
| | | ZFAND3 |
| | | CLN6 |
| | | KM-HN-1 |
| | | API5 |
| | | GPBP1L1 |
| | | PBOV1 |
| | | MRPL19 |
| | | GBP6 |
| | | TBL1XR1 |
| | | NT5C2 |
| | | SLC1A2 |
| | | ZC3H6 |
| | | PLXNC1 |
| | | PRKCBP1 |
| | | PRR3 |
| | | SEPN1 |
| | | ASCC3L1 |
| | | OR51E1 |
| | | HSDL2 |
| | | KIAA0141 |
| | | CENPJ |
| | | GJB2 |
| | | HOXB3 |
| | | OSGEP |
| | | WDR60 |
| | | ATP6AP2 |
| | | LDB1 |
| | | DENND1B |
| | | PAQR5 |
| | | SMCHD1 |
| | | EXT2 |
| | | ARHGAP30 |
| | | SLC22A16 |
| | | TRIM8 |
| | | CHST11 |
| | | PRMT2 |
| | | HEBP1 |
| | | GPR68 |
| | | CEP78 |
| | | RGNEF |
| | | BAG2 |
| | | DKFZP686A01247 |
| | | ARHGAP27 |
| | | IPO11 |
| | | TSHZ2 |
| | | LOC339529 |
| | | GZMA |
| | | HIBADH |
| | | PPA2 |
| | | C14ORF2 |
| | | FLJ90709 |
| | | RBPMS |
| | | RHD |
| | | LIX1L |
| | | GJA1 |
| | | GALK2 |
| | | MSR1 |
| | | WDR37 |
| | | SFRS14 |
| | | SSH1 |
| | | KIF13A |
| | | EEA1 |
| | | RAPGEF6 |
| | | TIA1 |
| | | SPTAN1 |
| | | GNPTAB |
| | | PEX14 |
| | | TAP1 |
| | | ARSE |
| | | NT5DC1 |
| | | MED6 |
| | | GZMH |
| | | ARMCX1 |
| | | PTPRM |
| | | RRAGC |
| | | DFFA |
| | | FAM118B |
| | | ZKSCAN1 |
| | | C14ORF101 |
| | | RFC3 |
| | | SLC31A1 |
| | | MGP |
| | | MED31 |
| | | TBL1Y |
| | | RUFY3 |
| | | TAPBP |
| | | EEF1D |
| | | PBX1 |
| | | RASSF6 |
| | | ZBTB44 |
| | | HSD17B3 |
| | | ENTPD5 |
| | | LRCH1 |
| | | LOC149086 |
| | | SORBS1 |
| | | ZFY |
| | | TAF4B |
| | | PRKCE |
| | | ABP1 |
| | | HOXC4 |
| | | CD40 |
| | | TNNI3K |
| | | FAM53B |
| | | GAS1 |
| | | ETS1 |
| | | ZXDC |
| | | PGAP1 |
| | | EXOSC10 |
| | | FLVCR |
| | | MGC16703 |
| | | DDX19A |
| | | RARG |
| | | MIPOL1 |
| | | SH3RF1 |
| | | HSPA4 |
| | | CLDN8 |
| | | CATSPER2 |
| | | ZBTB26 |
| | | DEPDC6 |
| | | CSAD |
| | | GPR137B |
| | | LRP8 |
| | | NDUFA10 |
| | | PAPOLB |
| | | NUP133 |
| | | NAV2 |
| | | ZNF451 |
| | | DNAJC7 |
| | | PPAT |
| | | SUZ12 |
| | | PPP1R3E |
| | | RAB7L1 |
| | | CC2D1B |
| | | FBXL20 |
| | | GPC3 |
| | | RASSF5 |
| | | C9ORF93 |
| | | ZNF587 |
| | | GTF2I |
| | | CUL1 |
| | | DUSP6 |
| | | UTY |
| | | SMTNL2 |
| | | PTPN2 |
| | | TMPO |
| | | BHLHB9 |
| | | MPL |
| | | ZNF441 |
| | | SEC63 |
| | | KATNAL2 |
| | | NFIB |
| | | CLIC2 |
| | | GSTM5 |
| | | NAPE-PLD |
| | | ZNF638 |
| | | NAALAD2 |
| | | MGC57346 |
| | | SORL1 |
| | | DLEU1 |
| | | OSBPL1A |
| | | SLC44A5 |
| | | STEAP1 |
| | | ZNF169 |
| | | FRY |
| | | MAFF |
| | | WDR73 |
| | | CHES1 |
| | | SNHG10 |
| | | C7ORF44 |
| | | MALT1 |
| | | ZNF417 |
| | | C2ORF3 |
| | | DAPP1 |
| | | LOC644192 |
| | | DAPK1 |
| | | NUP210 |
| | | TMEM107 |
| | | ACPL2 |
| | | SYTL3 |
| | | SWAP70 |
| | | GLT25D2 |
| | | SNF1LK2 |
| | | APOE |
| | | C8ORF70 |
| | | KLKB1 |
| | | RXRB |
| | | PCNX |
| | | NID2 |
| | | NPTN |
| | | DMN |
| | | ARHGAP29 |
| | | SEC14L3 |
| | | CCDC69 |
| | | ABCA11 |
| | | RAB11FIP2 |
| | | DENND4C |
| | | FAS |
| | | NIPSNAP3A |
| | | SCAMP4 |
| | | TMEM17 |
| | | PTP4A3 |
| | | MEIS1 |
| | | ASXL1 |
| | | MORN1 |
| | | CHRNA9 |
| | | PATZ1 |
| | | RNF7 |
| | | TTBK2 |
| | | UBE2Q1 |
| | | DPYD |
| | | TOX |
| | | FBXO9 |
| | | SLC26A7 |
| | | PPP2R3A |
| | | ZNF148 |
| | | FUT11 |
| | | ATF6 |
| | | STK17B |
| | | SEC24A |
| | | TCP11L1 |
| | | ZNF663 |
| | | INE1 |
| | | DENND1A |
| | | GPR92 |
| | | CORO1A |
| | | FNBP1 |
| | | GPAM |
| | | CSPP1 |
| | | C6ORF153 |
| | | HIC2 |
| | | RUNDC2A |
| | | ITGB5 |
| | | TRIM5 |
| | | SNTB1 |
| | | LONRF2 |
| | | CROP |
| | | TNK2 |
| | | MOBKL2B |
| | | RPL37A |
| | | GRHL1 |
| | | RNF190 |
| | | BBS1 |
| | | PRDX3 |
| | | HINT1 |
| | | NUP160 |
| | | DLEU2 |
| | | CTPS2 |
| | | MIF4GD |
| | | DAP3 |
| | | ZC3H13 |
| | | GSTA2 |
| | | C6ORF123 |
| | | C8ORF38 |
| | | SEC22A |
| | | ROCK1 |
| | | ZFP14 |
| | | ABCC9 |
| | | ZNF177 |
| | | PCBD2 |
| | | RPA3 |
| | | TTC8 |
| | | C15ORF33 |
| | | RUFY2 |
| | | CYBRD1 |
| | | CEP135 |
| | | CCDC99 |
| | | C11ORF54 |
| | | LSM14B |
| | | ZNF711 |
| | | SLC23A2 |
| | | KLHDC1 |
| | | H2AFY |
| | | SP4 |
| | | HTRA1 |
| | | NT5E |
| | | GRSF1 |
| | | GPR116 |
| | | ABCB11 |
| | | KNS2 |
| | | WHSC2 |
| | | ZNF703 |
| | | EFEMP2 |
| | | PLD3 |
| | | NEU4 |
| | | ALOX12 |
| | | MAD2L1 |
| | | PTPRF |
| | | MAPK1 |
| | | LOC389247 |
| | | ZNF740 |
| | | PAPOLA |
| | | NR1I2 |
| | | CATSPER3 |
| | | SLC40A1 |
| | | FAM48A |
| | | VPS13D |
| | | SMU1 |
| | | CYP7B1 |
| | | RUNX1T1 |
| | | NDUFS1 |
| | | RUNX3 |
| | | PPM1L |
| | | TAP2 |
| | | POLR2B |
| | | LGR4 |
| | | ELAVL1 |
| | | LIMK2 |
| | | TTN |
| | | GSTA4 |
| | | ALPK1 |
| | | C11ORF61 |
| | | ZNF2 |
| | | ARL6IP6 |
| | | SNX25 |
| | | CFD |
| | | CNOT6L |
| | | MMEL1 |
| | | PERQ1 |
| | | FIP1L1 |
| | | C9ORF150 |
| | | CRSP2 |
| | | SSBP2 |
| | | TDRD6 |
| | | PSMD7 |
| | | ZCCHC7 |
| | | EIF4B |
| | | ITSN2 |
| | | TFAP2A |
| | | KLHL22 |
| | | HSD17B12 |
| | | C21ORF59 |
| | | SH2D1A |
| | | PDGFC |
| | | PLXND1 |
| | | NEXN |
| | | PECI |
| | | DCAKD |
| | | FLJ36840 |
| | | B2M |
| | | GK5 |
| | | ECHDC2 |
| | | ZNF564 |
| | | SNED1 |
| | | ZNF781 |
| | | JMJD1C |
| | | HCRP1 |
| | | AGPAT1 |
| | | CRYL1 |
| | | DNAJC5B |
| | | TF |
| | | NAP1L5 |
| | | TSPYL1 |
| | | PAPPA |
| | | NAG6 |
| | | GBP1 |
| | | MAWBP |
| | | RORA |
| | | RAB33A |
| | | C9ORF80 |
| | | JPH1 |
| | | LRFN1 |
| | | LRRC17 |
| | | GRK4 |
| | | HNRPH3 |
| | | ZNF615 |
| | | TBC1D22B |
| | | SSPN |
| | | NUP43 |
| | | CFDP1 |
| | | PIGL |
| | | CNOT4 |
| | | MZF1 |
| | | VGLL3 |
| | | RNASEH2B |
| | | KIAA1727 |
| | | LRRIQ1 |
| | | MED28 |
| | | FYN |
| | | HMGCS1 |
| | | C1ORF210 |
| | | TMEM116 |
| | | TCEAL2 |
| | | CAPNS2 |
| | | TFDP2 |
| | | LOC286189 |
| | | SORT1 |
| | | MPP6 |
| | | TTC27 |
| | | MOSC2 |
| | | AFF4 |
| | | CENTD1 |
| | | CAND1 |
| | | HSPA4L |
| | | RNF166 |
| | | FAM125B |
| | | PPP3R1 |
| | | NFIC |
| | | CRLS1 |
| | | ZNF677 |
| | | ITGA2 |
| | | CPNE8 |
| | | C12ORF62 |
| | | RABEP1 |
| | | CEP192 |
| | | TLR3 |
| | | RAD50 |
| | | STK32C |
| | | ZSWIM5 |
| | | WFDC2 |
| | | SLAMF8 |
| | | SSH2 |
| | | PFAAP5 |
| | | PLEK2 |
| | | C4ORF29 |
| | | C6ORF201 |
| | | TMEM133 |
| | | LOC441461 |
| | | GCLM |
| | | CH25H |
| | | SLIT1 |
| | | SF3A1 |
| | | NFRKB |
| | | PTGER4 |
| | | ABCA9 |
| | | CALCA |
| | | TYSND1 |
| | | LOC644246 |
| | | PRR5 |
| | | MANEA |
| | | ST18 |
| | | MLLT10 |
| | | FLJ38717 |
| | | P2RY5 |
| | | MEIS2 |
| | | C1ORF125 |
| | | ARHGAP9 |
| | | CSMD1 |
| | | CCDC8 |
| | | KIAA2026 |
| | | ITGA9 |
| | | CD1C |
| | | ISL1 |
| | | ZMYM5 |
| | | SHANK2 |
| | | CIRBP |
| | | RHOBTB2 |
| | | SP3 |
| | | NMT2 |
| | | RSAD2 |
| | | NAPSB |
| | | CANX |
| | | ZNF251 |
| | | LOC387647 |
| | | FXC1 |
| | | KBTBD6 |
| | | WDFY3 |
| | | FMNL3 |
| | | ZC3H12C |
| | | TCP11 |
| | | ZNF562 |
| | | MDM4 |
| | | TRIM73 |
| | | CDKL2 |
| | | ZNF345 |
| | | TRPM7 |
| | | WASPIP |
| | | ZBED1 |
| | | UNC84A |
| | | NEK3 |
| | | ZNF83 |
| | | ZNF418 |
| | | NMNAT2 |
| | | USP6 |
| | | MAPKBP1 |
| | | ACVR1B |
| | | KIAA0286 |
| | | STARD7 |
| | | INSR |
| | | OR2A4 |
| | | CYP2U1 |
| | | MYO1D |
| | | CES4 |
| | | CCL20 |
| | | C1QTNF3 |
| | | LIG3 |
| | | MFAP3L |
| | | BCL9 |
| | | ZNF224 |
| | | CHFR |
| | | MRPL35 |
| | | LOC147804 |
| | | APOL6 |
| | | GNPDA2 |
| | | ZNF292 |
| | | SCFD1 |
| | | CMAH |
| | | EFNA4 |
| | | PPIL4 |
| | | KLRB1 |
| | | MATR3 |
| | | GULP1 |
| | | KCNJ1 |
| | | ELAC1 |
| | | RAPGEF5 |
| | | HIST1H2BC |
| | | ARHGEF12 |
| | | TNS3 |
| | | LRRN3 |
| | | TRAM1L1 |
| | | JUB |
| | | GNG2 |
| | | NEB |
| | | CXADR |
| | | FMN1 |
| | | ADHFE1 |
| | | FAM82B |
| | | MFAP3 |
| | | ZNF141 |
| | | SLC28A2 |
| | | CDC2L5 |
| | | BEX1 |
| | | FAM122C |
| | | CASP8 |
| | | ZBTB20 |
| | | SLC4A4 |
| | | PIAS1 |
| | | EXOC6 |
| | | KIAA0090 |
| | | FOXP1 |
| | | ANGPT2 |
| | | C6ORF162 |
| | | ASPHD1 |
| | | DIP2C |
| | | C5ORF24 |
| | | ANKS6 |
| | | YME1L1 |
| | | HNRPD |
| | | AGBL3 |
| | | EEF1G |
| | | INSM1 |
| | | MALL |
| | | ELMOD2 |
| | | IGSF10 |
| | | SYT9 |
| | | TSGA14 |
| | | FN3K |
| | | COL5A2 |
| | | UGT1A6 |
| | | CKMT2 |
| | | CAPS2 |
| | | ARPC4 |
| | | FMO4 |
| | | S100A7 |
| | | C12ORF29 |
| | | CRBN |
| | | SMYD2 |
| | | IGF2R |
| | | RASA2 |
| | | JAM3 |
| | | CFTR |
| | | CDKN2B |
| | | CYP4F11 |
| | | EDG2 |
| | | BCMO1 |
| | | FLJ36644 |
| | | C8ORF50 |
| | | ZNF19 |
| | | PSMB9 |
| | | TBCA |
| | | ITM2A |
| | | GTF2A2 |
| | | KLF15 |
| | | MGST1 |
| | | CWF19L2 |
| | | LMTK2 |
| | | RAD23B |
| | | PITPNB |
| | | ZNF131 |
| | | KCTD7 |
| | | SH3YL1 |
| | | SNRPN |
| | | IRF5 |
| | | MKL2 |
| | | KIAA1199 |
| | | MYO5A |
| | | ASB4 |
| | | SPESP1 |
| | | EDC3 |
| | | GPR115 |
| | | NCDN |
| | | SLC26A9 |
| | | SLC6A20 |
| | | HBS1L |
| | | MPHOSPH6 |
| | | PSMB7 |
| | | ZDHHC21 |
| | | P11 |
| | | CTSW |
| | | GPR98 |
| | | RTN1 |
| | | ABCC6 |
| | | SATB1 |
| | | LRCH3 |
| | | MAGI2 |
| | | FKBP1A |
| | | SCYL1BP1 |
| | | CD247 |
| | | TRIM13 |
| | | C1GALT1C1 |
| | | CYP2B6 |
| | | SNF8 |
| | | BIN3 |
| | | CLGN |
| | | KIAA1704 |
| | | DPY19L2 |
| | | SFRS15 |
| | | C14ORF132 |
| | | AKAP8L |
| | | ATP1B1 |
| | | GLS |
| | | EHBP1L1 |
| | | PPM2C |
| | | BCAS1 |
| | | ST3GAL5 |
| | | RPUSD3 |
| | | MEG3 |
| | | PISD |
| | | NBR2 |
| | | WTAP |
| | | SFXN3 |
| | | LOC641298 |
| | | C10ORF6 |
| | | GOSR2 |
| | | SMPD3 |
| | | DMXL2 |
| | | KIAA1276 |
| | | GPR177 |
| | | CCR6 |
| | | SEC22B |
| | | EIF3S9 |
| | | CDON |
| | | ISLR |
| | | SLC13A3 |
| | | SYNCRIP |
| | | ZNF160 |
| | | GGTL3 |
| | | CPT1B |
| | | ZNF230 |
| | | SBK1 |
| | | IFI6 |
| | | MAP3K3 |
| | | LRMP |
| | | FLJ36874 |
| | | ZNF382 |
| | | HEXA |
| | | TMED10 |
| | | FLCN |
| | | LSM6 |
| | | PDCD5 |
| | | C10ORF113 |
| | | CEP63 |
| | | GTPBP10 |
| | | TAS2R4 |
| | | ADAM17 |
| | | DRAM |
| | | RNF182 |
| | | IFT88 |
| | | RERE |
| | | SCMH1 |
| | | MAP2 |
| | | SPRY1 |
| | | SFTPB |
| | | LOC572558 |
| | | GPM6B |
| | | DECR1 |
| | | DAB1 |
| | | ZRANB3 |
| | | HSPA2 |
| | | TMEM161B |
| | | HP1BP3 |
| | | HFE |
| | | WNT2B |
| | | GPR89A |
| | | MAP3K15 |
| | | CYP51A1 |
| | | SIAH1 |
| | | RCOR3 |
| | | SPARCL1 |
| | | EFNA5 |
| | | KIAA1900 |
| | | GRM5 |
| | | ERN1 |
| | | CHURC1 |
| | | ZNF551 |
| | | DSPP |
| | | STAU2 |
| | | ZNF502 |
| | | RDX |
| | | CRISPLD2 |
| | | PDE5A |
| | | PIK3C2B |
| | | ZFR |
| | | TCP10L |
| | | IRAK2 |
| | | SLC2A13 |
| | | NT5C1B |
| | | CDC42SE2 |
| | | SLC1A4 |
| | | KIAA0892 |
| | | CTAGE5 |
| | | GGT6 |
| | | CAB39L |
| | | SDK1 |
| | | WDR44 |
| | | MKLN1 |
| | | WHSC1L1 |
| | | DOCK7 |
| | | GBP5 |
| | | PDPN |
| | | FLJ12529 |
| | | HIST1H1E |
| | | TRIM69 |
| | | L3MBTL4 |
| | | CDC42SE1 |
| | | CORO2B |
| | | GRLF1 |
| | | DDI2 |
| | | SOX30 |
| | | DNAJA5 |
| | | SERPINA6 |
| | | LOC389765 |
| | | MED4 |
| | | FOXQ1 |
| | | PLTP |
| | | CKMT1A |
| | | SLC1A1 |
| | | ZNF749 |
| | | PIP5K1B |
| | | ANKRD31 |
| | | ACSL4 |
| | | MPP7 |
| | | ADCK2 |
| | | LMBRD2 |
| | | DKK3 |
| | | ARL6IP2 |
| | | STAT4 |
| | | TTLL6 |
| | | HSF1 |
| | | WNT5B |
| | | LMO4 |
| | | ZNF785 |
| | | SLC6A6 |
| | | FHOD3 |
| | | CCL28 |
| | | LOC344595 |
| | | FGF7 |
| | | AUH |
| | | IGFBP5 |
| | | P15RS |
| | | PRDM16 |
| | | KIAA1909 |
| | | EIF2C3 |
| | | GOLGA1 |
| | | PDLIM4 |
| | | RAB30 |
| | | FANCC |
| | | CHL1 |
| | | BMP2K |
| | | TBC1D8 |
| | | SLC13A4 |
| | | ENPP3 |
| | | LOC157860 |
| | | MICAL3 |
| | | FLJ12334 |
| | | ABCA13 |
| | | RBBP4 |
| | | ELL2 |
| | | OXCT2 |
| | | UBE2Z |
| | | MAGEE1 |
| | | ITIH2 |
| | | NXT1 |
| | | LRRFIP2 |
| | | C21ORF62 |
| | | DKFZP667F0711 |
| | | LOC339874 |
| | | HLF |
| | | TM2D1 |
| | | CD2 |
| | | IKZF5 |
| | | FGFR1OP2 |
| | | SCG2 |
| | | SGCB |
| | | NBR1 |
| | | CDH12 |
| | | SERPING1 |
| | | ALX3 |
| | | PITX1 |
| | | GAB2 |
| | | TAGLN3 |
| | | DKFZP779O175 |
| | | SOS1 |
| | | GALM |
| | | ATP6V0C |
| | | LOC284412 |
| | | ADCY2 |
| | | ZRANB2 |
| | | HSMPP8 |
| | | TSPAN12 |
| | | ERICH1 |
| | | ARHGAP26 |
| | | PCNP |
| | | PCYOX1 |
| | | PTPRZ1 |
| | | CSF3 |
| | | QSCN6L1 |
| | | FCHSD2 |
| | | ETNK1 |
| | | PDCD2 |
| | | PLK2 |
| | | ZNF570 |
| | | KIAA1429 |
| | | RBMX |
| | | SCML1 |
| | | UBE3A |
| | | ANAPC5 |
| | | SNRPA1 |
| | | IL17RD |
| | | ACADL |
| | | GSTM4 |
| | | C11ORF47 |
| | | ZNF606 |
| | | MMRN2 |
| | | DOC1 |
| | | AR |
| | | INPP5D |
| | | NRXN3 |
| | | FAM83H |
| | | PRKXP1 |
| | | MAX |
| | | C11ORF30 |
| | | ELF2 |
| | | SLC16A6 |
| | | KIF2A |
| | | CAMK2D |
| | | TRIM33 |
| | | CLASP2 |
| | | AK2 |
| | | CHN2 |
| | | C14ORF145 |
| | | CRLF3 |
| | | DOPEY1 |
| | | AASS |
| | | EML1 |
| | | ANKH |
| | | MAN1C1 |
| | | ASAHL |
| | | FGFR2 |
| | | MAP3K7IP2 |
| | | C21ORF128 |
| | | RAD51L1 |
| | | PRPF18 |
| | | PLA1A |
| | | DNAH14 |
| | | INTS10 |
| | | BRWD1 |
| | | PKN2 |
| | | RABGAP1 |
| | | GGPS1 |
| | | USP51 |
| | | KCNN3 |
| | | PDE8A |
| | | DEXI |
| | | RCBTB1 |
| | | MGC61598 |
| | | HLA-DPB1 |
| | | C8ORF46 |
| | | PCDHB5 |
| | | FLJ11827 |
| | | COPS3 |
| | | SARS |
| | | PARVA |
| | | HSPA12A |
| | | GPNMB |
| | | KIAA1274 |
| | | P4HA2 |
| | | CD69 |
| | | APOD |
| | | DUSP16 |
| | | CTNNB1 |
| | | CG012 |
| | | LOC400927 |
| | | C8ORF34 |
| | | PVRL3 |
| | | IL6ST |
| | | SR140 |
| | | ZDHHC2 |
| | | SEC14L4 |
| | | SCG5 |
| | | SCML4 |
| | | CCDC76 |
| | | BCL2L11 |
| | | CXCL2 |
| | | FAM80B |
| | | SYT13 |
| | | LCMT2 |
| | | VNN1 |
| | | FST |
| | | FUT4 |
| | | DHX29 |
| | | XPA |
| | | POLR1D |
| | | NUMB |
| | | AMT |
| | | ABHD13 |
| | | RFT1 |
| | | BCL11A |
| | | C6ORF125 |
| | | AADAT |
| | | TCF20 |
| | | EML4 |
| | | DNASE1L3 |
| | | PTHB1 |
| | | ARMCX4 |
| | | CPSF6 |
| | | CYLD |
| | | VPS13A |
| | | RNF130 |
| | | SCD |
| | | SERGEF |
| | | ZNF396 |
| | | WNK4 |
| | | ADRA2A |
| | | TRPC1 |
| | | RIF1 |
| | | EFNB3 |
| | | BCL2 |
| | | FCGBP |
| | | UEVLD |
| | | TCTE3 |
| | | CAV3 |
| | | CNTN3 |
| | | SCN4B |
| | | ALDH6A1 |
| | | PTPN22 |
| | | ZMAT1 |
| | | RPRM |
| | | NARG2 |
| | | PKD2 |
| | | SPON1 |
| | | CCND2 |
| | | LOC646903 |
| | | DCDC2 |
| | | VNN2 |
| | | IFNK |
| | | ERC1 |
| | | DPYSL2 |
| | | ODF1 |
| | | CNTD1 |
| | | MED12L |
| | | FTO |
| | | AQP4 |
| | | VPS35 |
| | | TGM2 |
| | | TLOC1 |
| | | PROSAPIP1 |
| | | CXCL3 |
| | | HLA-A |
| | | FXYD6 |
| | | EML5 |
| | | FLJ42627 |
| | | TCHP |
| | | EFHD1 |
| | | COX11 |
| | | KIAA1919 |
| | | ZNF506 |
| | | C17ORF63 |
| | | NR2C2 |
| | | CLK4 |
| | | PLEKHH1 |
| | | GPR135 |
| | | AMIGO2 |
| | | AXIN2 |
| | | ZDHHC13 |
| | | PRKRA |
| | | TMEM27 |
| | | LOC285628 |
| | | ANKRD44 |
| | | THRAP2 |
| | | ENPP2 |
| | | ACSL3 |
| | | SLC9A11 |
| | | BTN3A1 |
| | | EPAS1 |
| | | INOC1 |
| | | BCAM |
| | | NR1D2 |
| | | RAPH1 |
| | | EVL |
| | | CRISP3 |
| | | MTMR1 |
| | | DKFZP434C153 |
| | | CXCL6 |
| | | NLF2 |
| | | CCAR1 |
| | | NR3C2 |
| | | LOC400568 |
| | | LSM14A |
| | | KYNU |
| | | ZDHHC17 |
| | | ZNF154 |
| | | TMEM19 |
| | | CPVL |
| | | STEAP4 |
| | | AKAP11 |
| | | C4ORF19 |
| | | SMAD3 |
| | | AKAP7 |
| | | LOC221442 |
| | | PLXDC1 |
| | | DNMT3A |
| | | PEG3 |
| | | FRMD4A |
| | | FLJ13197 |
| | | FOXE1 |
| | | SCGB3A2 |
| | | TUB |
| | | NFATC2 |
| | | TTTY15 |
| | | SESN3 |
| | | LOC200772 |
| | | FBXL14 |
| | | ACE2 |
| | | KIAA0256 |
| | | NAALADL2 |
| | | ERO1LB |
| | | ME3 |
| | | SLC23A1 |
| | | SETBP1 |
| | | LOC285419 |
| | | GABBR1 |
| | | RAB3IP |
| | | ALKBH5 |
| | | ANXA6 |
| | | VMO1 |
| | | DAD1 |
| | | AKR1C2 |
| | | ZNF331 |
| | | BCL11B |
| | | STAT1 |
| | | D2HGDH |
| | | LOC283177 |
| | | VANGL2 |
| | | PCDH20 |
| | | MUC13 |
| | | FADS1 |
| | | HELLS |
| | | WNK2 |
| | | B4GALT6 |
| | | LGR6 |
| | | EFNB2 |
| | | LYN |
| | | C8ORF72 |
| | | ADAMTS8 |
| | | GOSR1 |
| | | STK17A |
| | | NKX3-1 |
| | | CD24 |
| | | FMNL2 |
| | | DSCR1L1 |
| | | LOC374443 |
| | | WDR42A |
| | | ANGEL2 |
| | | GABRE |
| | | FUBP1 |
| | | PPWD1 |
| | | GPR155 |
| | | SLC6A16 |
| | | C8ORF42 |
| | | PCDH7 |
| | | SHH |
| | | LONRF1 |
| | | PRKCA |
| | | SLC7A2 |
| | | TMOD2 |
| | | PRKCB1 |
| | | SUSD4 |
| | | SAA4 |
| | | USH1C |
| | | IFIT1 |
| | | GPRASP1 |
| | | BICC1 |
| | | HNMT |
| | | KLK10 |
| | | PRPF4 |
| | | HLA-DMB |
| | | IFI27 |
| | | FRAS1 |
| | | LOC200609 |
| | | ATF7IP |
| | | CREBBP |
| | | STEAP2 |
| | | HLA-DQB1 |
| | | GFI1 |
| | | EIF4ENIF1 |
| | | IFI44 |
| | | TRIM55 |
| | | HHLA2 |
| | | LOC493754 |
| | | NAV3 |
| | | PAX1 |
| | | ARNT |
| | | POLR3B |
| | | PER3 |
| | | BTN3A2 |
| | | KLF12 |
| | | C9ORF72 |
| | | NPNT |
| | | MYH15 |
| | | CYP4A11 |
| | | RNF152 |
| | | CCNF |
| | | HLA-F |
| | | SCGB3A1 |
| | | ELF5 |
| | | PCDH17 |
| | | GPR126 |
| | | WDR17 |
| | | KIF5C |
| | | MAOB |
| | | PNMA2 |
| | | ATP13A4 |
| | | CIITA |
| | | HLA-DMA |
| | | FMN2 |
| | | PPP1R9A |
| | | TFCP2 |
| | | SLC5A1 |
| | | ST8SIA4 |
| | | ITPR1 |
| | | TGFBR3 |
| | | CG018 |
| | | SLC15A2 |
| | | RAI2 |
| | | CLEC2D |
| | | TRAC |
| | | SUZ12P |
| | | CHGB |
| | | EPB41L5 |
| | | MTCH2 |
| | | PELO |
| | | GVIN1 |
| | | ZFP28 |
| | | CD1E |
| | | NOTCH2 |
| | | IL2RB |
| | | KIAA1450 |
| | | IRS2 |
| | | HLA-DQA1 |
| | | FLJ10781 |
| | | MGAT4A |
| | | SGCE |
| | | PLUNC |
| | | BTN3A3 |
| | | RIMS1 |
| | | FREM2 |
| | | RGN |
| | | HLA-DQB2 |
| | | PART1 |
| | | ITPR2 |
| | | IFI44L |
| | | SMARCA1 |
| | | LOC150005 |
| | | SCNN1G |
| | | C9ORF65 |
| | | EPB41L2 |
| | | ADAM28 |
| | | VNN3 |
| | | TGM3 |
| | | ZNF483 |
| | | P2RY2 |
| | | GATM |
| | | HMGA2 |
| | | LOC285045 |
| | | TTC7B |
| | | SLC39A10 |
| | | TNIK |
| | | LOC644135 |
| | | HLA-DPA1 |
| | | VEGF |
| | | TLL1 |
| | | SCUBE3 |
| | | SULF1 |
| | | ABCC4 |
| | | ATP8A1 |
| | | SKAP1 |
| | | GIT2 |
| | | NPAS3 |
| | | CXCR6 |
| | | WWC2 |
| | | SERPINB7 |
| | | RAB6B |
| | | LRP12 |
| | | ASCL1 |
| | | TMEM45A |
| | | RGS1 |
| | | PTGFR |
| | | CD3D |
| | | SLAMF7 |
| | | WNT5A |
| | | KCNMB2 |
| | | WIF1 |
| | | NELL2 |
| | | FLJ10038 |
| | | HLA-DPB2 |
| | | ZNF447 |
| | | TACR1 |
| | | RGS17 |
| | | PIP |
| | | KCNA1 |
| | | EPB41L3 |
| | | FCER1A |
| | | CD8A |
| | | FOXA2 |
| | | DPP4 |
| | | HEG1 |
| | | CA8 |
| | | CDH2 |
| | | CD3G |
| | | SKIP |
| | | CAPN13 |
| | | DOCK10 |
| | | GPR171 |
| | | CYP2A13 |
| | | HLA-DRB1 |
| | | PLAG1 |
| | | B3GALT5 |
| | | HLA-DOA |
| | | CCL5 |
| | | CXCR7 |
| | | SLC13A2 |
| | | KLRC4 /// KLRK1 |
| | | TMEM46 |
| | | PEG10 |
| | | GRP |

What is claimed is:

1. A method for assessing asthma risk status of a subject, preferably a human subject, the method comprising:
a) providing a saliva or blood sample from the subject;
b) assessing the RNA in the sample to determine the level of transcription of at least one gene selected from Table 1 or Table 2;
c) utilizing the results of (b) and (c) to characterize the subject according to their probable asthma status.

2. A method according to claim 1 wherein the method is utilized for the characterization of the subject according to their probable asthma status to establish a diagnostic intervention plan and/or treatment for the subject.

3. A method according to claim 1 wherein the method is utilized for classifying the subject according to their likelihood of having asthma status.

4. A method according to claim 1 wherein the method is utilized for classifying a population of subjects according to their likelihood of having asthma status.

5. A method according to claim 1 wherein the method is utilized for classifying the population of subjects according to their risk status and/or determining which subject of the population of subjects is at highest risk of developing asthma or severe asthma.

6. A method according to claim 1 wherein the method is utilized for classifying the subject according to the following risk status categories: (1) as low risk; (2) as a medium risk; and (3) as high risk of developing asthma.

7. A method according to claim 1 wherein the method is utilized for classifying the population according to the following risk status categories (1) as low risk, (2) as medium risk, and (3) as high risk of developing asthma.

8. A method according to claim 1 wherein the method is utilized to classify a healthy subject into category 1 characterized as low risk; or category 2 as a medium risk; or category 3 as high risk.

9. A method according to claim 1 wherein the method is utilized for assessing the risk of having or developing severe asthma in an asthmatic subject.

10. A method according to claim 1 wherein the method is aiding in the prognosis of asthma in the subject.

11. A method according to claim 1 wherein the method is utilized in the assessment of the efficacy of a treatment for asthma in the subject.

12. A method according to claim 1 wherein the method is utilized in monitoring the progression or regression of asthma in the subject.

13. A method according to claim 1 wherein the method is utilized to develop novel risk or prediction model based on RNA from salivary, blood sputum, brush, or biopsy markers.

14. A method according to claim 1 wherein the method is utilized to providing advice to a subject classified as being at or above low or medium risk in order to reduce the risk classification of the subject.

15. A method for assessing asthma risk status of a subject, preferably a human subject, the method comprising:
a) providing a saliva sample from the subject;
b) assessing the RNA in the sample to determine the level of transcription of at least one gene selected from Table 1 or Table 2;
c) utilizing the results of (b) and (c) to characterize the subject according to their probable asthma status;
d) utilizing the classification of the subject according to the following risk status categories: (1) as low risk;
(2) as a medium risk; and (3) as high risk of developing asthma to establish a diagnostic intervention plan and/or treatment plan for the subject.

16. A kit according to claim 1 comprising:
a) means for collecting saliva sample from the subject;
b) means for assessing the RNA in the sample in the sample to determine the level of transcription of at least one gene selected from Table 1 or Table 2;
c) utilizing the results of (b) and (c) to characterize the subject according to their probable asthma status.

17. A kit according to claim 16 wherein the method is utilized for the characterization of the subject according to their probable asthma status to establish a diagnostic intervention plan and/or treatment for the subject.

18. A kit according to claim 16 wherein the method is aiding in the prognosis of asthma in the subject.

19. A kit according to claim 16 wherein the method is utilized in monitoring the progression or regression of asthma in the subject.

20. A method of treating asthma in a subject comprising: providing a saliva sample from the subject;
a) assessing the RNA in the sample in the sample to determine the level of transcription of at least one gene selected from Table 1 or Table 2;
b) utilizing the results of (b) and (c) to characterize the subject according to their probable asthma status.
c) administering a specified treatment to the subject utilizing the characterization of the subject according to their probable asthma status.


SEQUENCE LISTING (9 sequences)

SEQ ID NO: 1 - DNA, 42 bp, Homo sapiens
gagcacctggaagcagtaacggatcacagcagacataaaggc

SEQ ID NO: 2 - DNA, 38 bp, Homo sapiens
gaagagctcctagcagtcgggccacttcttcattccag

SEQ ID NO: 3 - DNA, 44 bp, Homo sapiens
tattgtgaaggaggggatctggcgatgcaatacggtatgaccac

SEQ ID NO: 4 - DNA, 40 bp, Homo sapiens
gccattgaaacgatgccttggcaaaggctacaacacgttc

SEQ ID NO: 5 - DNA, 36 bp, Homo sapiens
cgacaagaagcgcatcatgtggtcttggccagagag

SEQ ID NO: 6 - DNA, 42 bp, Homo sapiens
gccccaaaaggaactaaaaggggatttcttgcttgtgactgc

SEQ ID NO: 7 - DNA, 39 bp, Homo sapiens
cgccagagcaggacgaaggacacatctgcatcctgaagt

SEQ ID NO: 8 - DNA, 40 bp, Homo sapiens
cttacacccaccatcccactcctccacaaattgctgctgt

SEQ ID NO: 9 - DNA, 19 bp, Homo sapiens
tgactcaacacgggaaacc